(19) United States
(12) Patent Application Publication
(10) Pub. No.: US 2013/0288967 A1
(43) Pub. Date: Oct. 31, 2013

(54) MATERIALS AND METHODS FOR CANCER DIAGNOSIS BY EVALUATION OF THE METHYLATION STATUS OF CPG ISLANDS ON CHROMOSOMES 6 AND 8

(71) Applicant: EUCLID DIAGNOSTICS LLC, CROWN POINT, IN (US)

(72) Inventors: Wadiha FREIJE, Forest Park, IL (US); Igor BRIKUN, Forest Park, IL (US); Deborah NUSSKERN, Forest Park, IL (US)

(73) Assignee: EUCLID DIAGNOSTICS LLC, CROWN POINT, IN (US)

(21) Appl. No.: 13/870,605

(22) Filed: Apr. 25, 2013

Related U.S. Application Data
(60) Provisional application No. 61/638,367, filed on Apr. 25, 2012.

(51) Int. Cl. C12Q 1/68

(52) U.S. Cl. CPC C12Q 1/6886
USPC 514/10.2; 435/6.11

(57) ABSTRACT

Provided are methods, reagents, and kits for evaluating cancer, such as prostate cancer, in a subject. Disclosed methods of evaluating cancer include methods of diagnosing cancer, methods of prognosticating cancer and methods of assessing the efficacy of cancer treatment. The methods include assaying a biological sample for methylation of a CpG island associated with specified genes. Provided reagents and kits include primers suitable for amplifying at least a portion of a target CpG islands associated with specified genes.

### CROSS-REFERENCE TO RELATED APPLICATIONS

This patent application claims the benefit of U.S. Provisional Patent Application No. 61/638,367, filed Apr. 25, 2012, which is incorporated by reference herein in its entirety.

### INCORPORATION-BY-REFERENCE OF MATERIAL ELECTRONICALLY SUBMITTED

Incorporated by reference in its entirety herein is a computer-readable nucleotide/amino acid sequence listing submitted concurrently herewith and identified as follows: 36,543 bytes ASCII (Text) file named “713395SequenceListing.txt,” created Apr. 24, 2013.

### BACKGROUND OF THE INVENTION

Phosphate linked cytosine-guanine (CpG) dinucleotides are statistically underrepresented in the genomes of higher eukaryotes, including mammals. The dinucleotide is reportedly found at only 5-10% of its predicted frequency. The majority of CpG dinucleotides that do remain in the human genome are normally located within repetitive sequences that are characterized by low gene expression levels and exhibit methylation at the cytosine residues.

CpG islands, on the other hand, represent genomic sequences that contain clusters of CpG dinucleotide. CpG islands may be associated with the promoter region or 5′ end of coding sequences or may be present within introns or in genomic regions that are not known to be associated with coding sequences. They may be unmethylated or methylated in normal tissues and the methylation pattern may be used to control tissue specific expression and the expression of imprinted genes. Methylation of CpG islands within promoter regions can result in the downregulation or silencing of the associated gene. An increase in methylation of normally unmethylated islands is observed in aging tissues even as the overall methylcytosine content of the DNA is reduced. The aberrant methylation pattern is more pronounced in cancer cells with increased methylation or hypermethylation detected in various cancer tissues. CpG islands may be methylated to varying densities within the same tissue. Thus, aberrant methylation of cytosines within CpG islands can be a primary epigenetic event that acts to suppress the expression of genes involved in critical cellular processes, such as DNA damage repair, hormone response, cell-cycle control, and tumor-cell adhesion/metastasis, leading to tumor initiation, progression and metastasis (Li et al., Biochim. Biophys. Acta, 1704: 87-102 (2004)). It has been proposed that a unique profile of promoter hypermethylation exists for each human cancer in which some gene changes are shared and other gene changes are cancer-type specific (Esteller et al., Cancer Res., 61: 3225-3229 (2001)). Given that aberrant methylation represents new information not normally present in genomic DNA and that aberrant methylation is a common DNA modification and affects a large number of genomic targets, it is feasible to develop diagnostic and prognostic tests based on information obtained from multiple target CpGs. Such tests may be based on CpGs that are aberrantly hypermethylated or hypomethylated in the diseased tissues. They may also be based on changes in methylation density in CpG islands as long as the changes correlate with the presence of cancer.

Prostate cancer, for example, which is the most common malignancy and the second leading cause of death among men in the U.S. (Li et al. (2004), supra), has been found to be associated with the methylation of CpG islands in the promoters of over 30 genes, in particular the CpG island of the glutathione S-transferase P1 (GSTP1) gene. GSTP1 methylation has been detected in over 50% of DNA recovered from urine and plasma of prostate cancer patients (Goessl et al., Ann. N.Y. Acad. Sci., 945: 51-58 (2001); Cairns et al., Clin. Cancer Res., 7: 2727-2730 (2001); Jeronimo et al., Urology, 60: 1131-1135 (2002); and Gonzalgo et al., Clin. Cancer Res., 9: 2673-2677 (2003)). However, if diagnosis of prostate cancer relied solely on the detection of the methylation of the CpG island in the GSTP1 gene, the theoretical limit of the sensitivity of such a test would only be approximately 90%. GSTP1 is also methylated in prostatic intraepithelial lesions (PIN) which may lead to a false positive diagnosis. Some CpG islands are methylated in prostate cancer and other diseases of the prostate, such as benign prostatic hyperplasia (BPH). They may even exhibit some degree of methylation in normal aging prostates. Such markers may not be suitable individually for prostate cancer diagnosis. Therefore, a panel of markers is required to achieve the sensitivity and specificity needed for a clinical test.

The prostate-specific antigen or PSA test continues to be widely used in the early detection of prostate cancer. While the PSA test has resulted in the majority of prostate cancer cases being diagnosed in asymptomatic men (Mettlin et al., Cancer, 83(8): 1679-1684 (1998a); Mettlin et al., Cancer, 82(2): 249-251 (1998b); Humphrey et al., J. Urol., 155: 816-820 (1996); and Grossfeld et al., Epidemiol. Rev., 23(1): 173-180 (2001)), the PSA test suffers from poor specificity, which can be as low as 33% when a PSA cut-off level of 2.6 ng/ml is used (Thompson et al., N. Engl. J. Med., 350: 2239-2246 (2004)), even though the sensitivity can be as high as 83%. The poor specificity of the PSA test is a direct result of increased secretion of PSA in other diseases of the prostate, such as BPH and prostatitis. Thus, an elevated PSA level indicates the need for additional screening in the form of needle biopsy. Ultimately, the results of needle biopsies lead to the diagnoses of prostate cancer.

Over 1 million needle biopsies of prostates are performed each year at a cost of about $1,500 each and much discomfort to the patient. However, less than 200,000 of these result in a diagnosis of prostate cancer. Therefore, the majority of needle biopsies are being performed needlessly.

In view of the above, there is a need for non-invasive methods of diagnosing and prognosticating cancer, such as prostate cancer, that reduce the cost and suffering associated with currently available cancer screening methods. It is an object of the invention to provide materials and methods for non-invasive diagnosis and prognosis of cancer, such as prostate cancer. This and other objects and advantages, as well as additional inventive features, will become apparent from the detailed description provided herein.

### BRIEF SUMMARY OF THE INVENTION

The invention provides materials and methods for evaluating and treating cancer including prostate cancer. The invention is based, at least in part, on the discovery that the methylation status of CpG islands associated with genes on chromosome 6 and chromosome 8 are diagnostic and prognostic of cancer. Thus, the invention provides methods of diagnosing and prognosticating cancer as well as methods of assessing the efficacy of cancer treatment. The invention further provides methods of treating cancer based on the disclosed methods of diagnosing and assessing the efficacy of cancer treatment.

Generally, the methods provided herein involve assaying for methylation status of one or more CpG islands associated with genes located on chromosomes 6 and/or 8. The invention also provides pairs of isolated, purified, or synthesized primers that can be used to assay for the methylation status of one or more CpG islands associated with genes on chromosome 6 and/or chromosome 8. The primers can be used, for example, to amplify and/or detect the methylation status of the one or more CpG islands. The invention also provides kits comprising one or more pairs of primers useful in the disclosed methods.

The invention provides a method of diagnosing cancer, e.g., prostate cancer, by assaying for one or more methylated CpG islands that are indicative of cancer. Generally, the method comprises providing a biological sample from a subject in need of cancer diagnosis and assaying the sample for methylation of one or more CpG islands associated with at least one gene selected from the group of: ARHGEF10 (Rho guanine nucleotide exchange factor 10 gene), NT5E (5′-nucleotidase, ecto (CD73) gene), MOXD1 (monooxygenase, DBH-like 1 gene), PTPRK (protein tyrosine phosphatase, receptor type K gene), and PHIP (pleckstrin homology domain interacting protein gene). ARHGEF10 is located on chromosome 8. NT5E, MOXD1, PTPRK, and PHIP are located on chromosome 6.

The invention also provides a method of diagnosing prostate cancer in a male mammal by assaying for one or more methylated CpG islands that are indicative of prostate cancer. The method can include providing a biological sample from a subject in need of prostate cancer diagnosis, e.g., a subject undergoing prostate cancer evaluation, and assaying the sample for methylation of one or more CpG islands associated with at least one gene selected from the group consisting of: ARHGEF10, NT5E, MOXD1, PTPRK, and PHIP. Optionally, the method of diagnosing prostate cancer can also include assaying for methylation of one or more CpG islands associated with at least one gene that is known to be methylated in prostate cancer but is known not to be detectably methylated or is methylated at a lower level (e.g., about 50% or less, about 40% or less, 30% or less, about 20% or less, or about 10% or less) in BPH. The invention further provides a method of treating cancer based on the foregoing diagnosis. When the biological sample includes one or more methylated CpG islands associated with at least one gene selected from the group of: ARHGEF10, NT5E, MOXD1, PTPRK, and PHIP, the subject can be treated for prostate cancer.

The invention also provides methods of prognosticating cancer by assaying for the methylation of one or more genes that are indicative of the grade or stage of the cancer, and/or the length of disease-free survival following treatment for cancer. Generally, the method comprises providing a biological sample from a subject in need of cancer prognosis and assaying the sample for methylation of one or more CpG islands associated with at least one gene selected from the group consisting of: ARHGEF10, NT5E, MOXD1, PTPRK, and PHIP.

Optionally, the method of prognosticating prostate cancer can also include assaying the biological sample for methylation of one or more CpG islands associated with at least one gene that is known to be methylated in prostate cancer but is known not to be detectably methylated or is methylated at a lower level (e.g., about 50% or less, about 40% or less, 30% or less, about 20% or less, or about 10% or less) in BPH. Methylation of the CpG islands associated with the genes is indicative of the grade or stage of the cancer, and/or the length of disease-free survival following treatment.

Furthermore, the invention provides methods of assessing the efficacy of treatment of cancer by assaying for the reduced methylation of CpG islands that indicates efficacy of treatment. Generally, the method comprises providing a first and a second biological sample from a subject in need of assessing the efficacy of treatment of cancer and assaying the samples for a change in the levels of methylation of one or more CpG islands associated with at least one gene selected from the group of: ARHGEF10, NT5E, MOXD1, PTPRK, and PHIP. The first biological sample is taken before the second biological sample, and the second biological sample is taken during or after a course of cancer treatment. A decrease or absence of methylation of the assayed CpG islands in the second sample (following treatment) indicates that the treatment is effective. Alternatively, the maintenance or increase of methylation in the assayed CpG islands in the second sample can indicate a reduction or absence of treatment efficacy.

Also provided is a method of assessing the efficacy of treatment of prostate cancer in a male mammal by assaying biological samples, which are taken from the male mammal periodically during the course of treatment, for methylation of one or more CpG islands and wherein a decrease or absence of methylation of the CpG islands following the course of treatment indicates that the treatment is effective. The method comprises (a) providing a first and a second biological sample from a subject undergoing a course of prostate cancer treatment, wherein the first sample is taken at an earlier time than the second sample, and the second sample is taken during or following a course of treatment and (b) assaying the samples for methylation of one or more CpG islands associated with at least one gene selected from the group consisting of: ARHGEF10, NT5E, MOXD1, PTPRK, and PHIP. A decrease or absence of methylation of the assayed one or more CpG islands in the second sample (i.e., following a treatment) indicates that the prostate cancer treatment is effective for the subject. Alternatively, the maintenance or increase of methylation in the assayed CpG islands in the second sample can indicate that the treatment has reduced efficacy or has no efficacy for the subject. Optionally, this method can also include assaying the biological samples for methylation of one or more CpG islands associated with at least one gene that is known to be methylated in prostate cancer but is known not to be detectably methylated or is methylated at a lower level (e.g. about 50% or less, about 40% or less, 30% or less, about 20% or less, or about 10% or less) in BPH. The invention further provides a method of treating cancer based on the foregoing assessment of treatment efficacy. When the foregoing assessment indicates that treatment is efficacious for the subject, the subject can continue the same course of prostate cancer treatment or cease further treatment. Alternatively, when the foregoing assessment indicates that treatment is not efficacious for the subject, the subject can include additional or different treatment for prostate cancer. Additional treatments can include further surgical interventions or increased dose of the same medications. A different treatment can include a different type of surgical intervention, or a different type of medication, e.g., radiation therapy.

In preferred embodiments, the aforementioned methods of diagnosing, prognosticating and assessing the efficacy of treatment of cancer can further include assaying the biological sample for methylation of multiple CpG islands, for example, CpG islands associated with two, three, four, or all five genes selected from the group of: ARHGEF10, NTSE, MOXD1, PTPRK, and PHIP.

The invention also provides pairs of primers suitable for amplifying a CpG-island associated with genes described herein. Primers can include isolated, purified, or synthetic nucleic acid molecules suitable for amplifying a CpG island containing target sequence. Target sequences can include genomic sequence that is fully methylated, partially methylated, and fully unmethylated. Target sequences also include methylated, partially methylated, and fully unmethylated sequences after treatment with a deaminating agent, such as sodium bisulfite (“bisulfite treatment”). Exemplary target sequences are provided herein for ARHGEF10, NTSE, MOXD1, PTPRK, and PHIP are provided herein and include SEQ ID NOs: 1 to 15. Exemplary primers for amplifying these sequences are also provided herein.

Also provided are kits that include one or more of the aforementioned pairs of primers.

### BRIEF DESCRIPTION OF THE FIGURES

FIG. 1 shows an exemplary genomic target region sequence associated with ARHGEF10 (SEQ ID NO: 1). The sequence can be fully CpG methylated, partially CpG methylated, or non-methylated at CpG.

FIG. 2 shows the sequence (SEQ ID NO: 2) resulting from the bisulfite treatment of the exemplary genomic target region sequence associated with ARHGEF10 (SEQ ID NO: 1), which was fully CpG methylated prior to bisulfite treatment.

FIG. 3 shows the sequence (SEQ ID NO: 3) resulting from the bisulfite treatment of the exemplary genomic target region sequence associated with ARHGEF10 (SEQ ID NO: 1), which was fully non-methylated at CpG prior to bisulfite treatment.

FIGS. 4A and 4B show an alignment of an exemplary genomic target region sequence associated with ARHGEF10 (SEQ ID NO: 1) (top row sequence), the sequence resulting from bisulfite treatment of ARHGEF10 which was fully methylated prior to bisulfite treatment (SEQ ID NO: 2) (middle row sequence), and the sequences resulting from bisulfite treatment of ARHGEF10 which was fully non-methylated prior to bisulfite treatment (SEQ ID NO: 3) (bottom row sequence).

FIG. 5 shows an exemplary genomic target region sequence associated with MOXD1 (SEQ ID NO: 4). The sequence can be fully CpG methylated, partially CpG methylated, or non-methylated at CpG.

FIG. 6 shows the sequence (SEQ ID NO: 5) resulting from the bisulfite treatment of the exemplary genomic target region sequence associated with MOXD1 (SEQ ID NO: 4), which was fully CpG methylated prior to bisulfite treatment.

FIG. 7 shows the sequence (SEQ ID NO: 6) resulting from the bisulfite treatment of the exemplary genomic target region sequence associated with MOXD1 (SEQ ID NO: 4), which was fully non-methylated at CpG prior to bisulfite treatment.

FIGS. 8A and 8B show an alignment of an exemplary genomic target region sequence associated with MOXD1 (SEQ ID NO: 4) (top row sequence), the sequence resulting from bisulfite treatment of MOXD1 which was fully methylated prior to bisulfite treatment (SEQ ID NO: 5) (middle row sequence), and the sequence resulting from bisulfite treatment of MOXD1 which was fully non-methylated prior to bisulfite treatment (SEQ ID NO: 6) (bottom row sequence).

FIG. 9 shows an exemplary genomic target region sequence associated with NT5E (SEQ ID NO: 7). The sequence can be fully CpG methylated, partially CpG methylated, or non-methylated at CpG.

FIG. 10 shows the sequence (SEQ ID NO: 8) resulting from the bisulfite treatment of the exemplary genomic target region sequence associated with NT5E (SEQ ID NO: 7), which was fully CpG methylated prior to bisulfite treatment.

FIG. 11 shows the sequence (SEQ ID NO: 9) resulting from the bisulfite treatment of the exemplary genomic target region sequence associated with NT5E (SEQ ID NO: 7), which was fully non-methylated at CpG prior to bisulfite treatment.

FIGS. 12A and 12B show an alignment of an exemplary genomic target region sequence associated with NT5E (SEQ ID NO: 7) (top row sequence), the sequence resulting from bisulfite treatment of NT5E which was fully methylated prior to bisulfite treatment (SEQ ID NO: 8) (middle row sequence), and the sequence resulting from bisulfite treatment of NT5E which was fully non-methylated prior to bisulfite treatment (SEQ ID NO: 9) (bottom row sequence).

FIGS. 13A and 13B show an exemplary genomic target region sequence associated with PHIP (SEQ ID NO: 10). The sequence can be fully CpG methylated, partially CpG methylated, or non-methylated at CpG.

FIGS. 14A and 14B show the sequence (SEQ ID NO: 11) resulting from the bisulfite treatment of the exemplary genomic target region sequence associated with PHIP (SEQ ID NO:10), which was fully CpG methylated prior to bisulfite treatment.

FIGS. 15A and 15B show the sequence (SEQ ID NO: 12) resulting from the bisulfite treatment of the exemplary genomic target region sequence associated with PHIP (SEQ ID NO: 10), which was fully non-methylated at CpG prior to bisulfite treatment.

FIGS. 16A, 16B, and 16C show an alignment of an exemplary genomic target region sequence associated with PHIP (SEQ ID NO: 10) (top row sequence), the sequence resulting from bisulfite treatment of PHIP which was fully methylated prior to bisulfite treatment (SEQ ID NO: 11) (middle row sequence), and the sequence resulting from bisulfite treatment of PHIP which was fully non-methylated prior to bisulfite treatment (SEQ ID NO: 12) (bottom row sequence).

FIGS. 17A and 17B show an exemplary genomic target region sequence associated with PTPRK (SEQ ID NO: 13). The sequence can be fully CpG methylated, partially CpG methylated, or non-methylated at CpG.

FIGS. 18A and 18B show the sequence (SEQ ID NO: 14) resulting from the bisulfite treatment of the exemplary genomic target region sequence associated with PTPRK (SEQ ID NO: 13), which was fully CpG methylated prior to bisulfite treatment.

FIGS. 19A and 19B show the sequence (SEQ ID NO: 15) resulting from the bisulfite treatment of the exemplary genomic target region sequence associated with PTPRK (SEQ ID NO: 13), which was fully non-methylated at CpG prior to bisulfite treatment.

FIGS. 20A, 20B, and 20C show an alignment of an exemplary genomic target region sequence associated with PTPRK (SEQ ID NO: 13) (top row sequence), the sequence resulting from bisulfite treatment of PTPRK which was fully methylated prior to bisulfite treatment (SEQ ID NO: 14) (middle row sequence), and the sequence resulting from bisulfite treatment of PTPRK which was fully non-methylated prior to bisulfite treatment (SEQ ID NO: 15) (bottom row sequence).

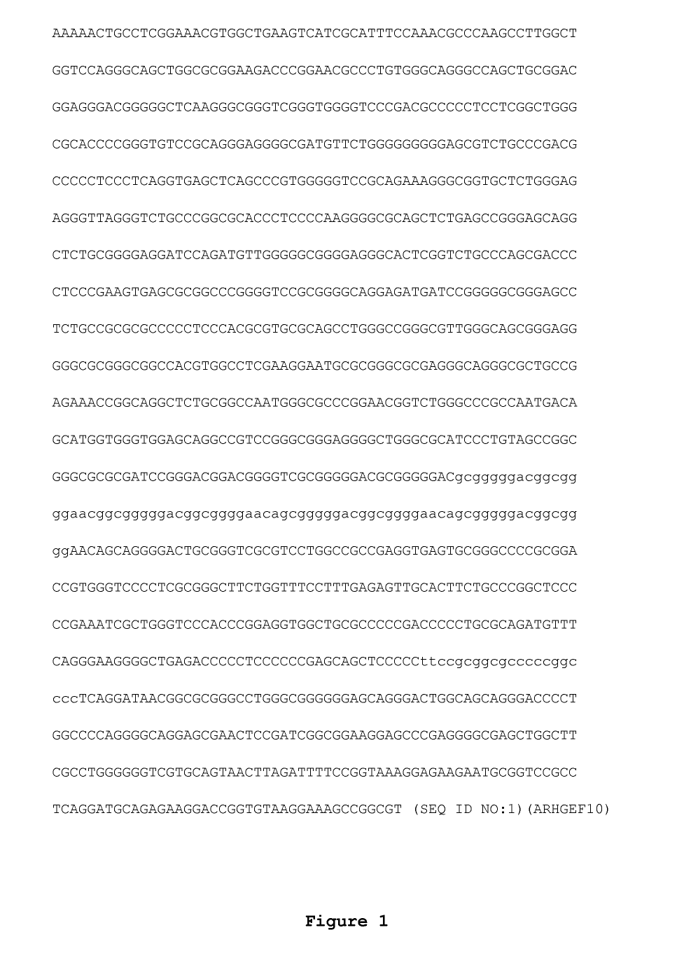

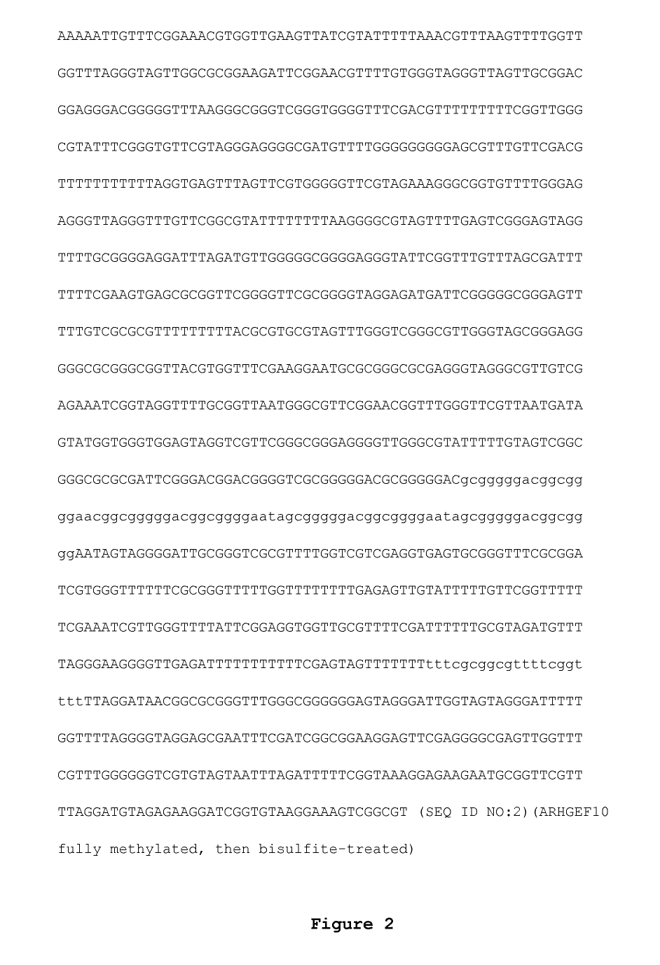

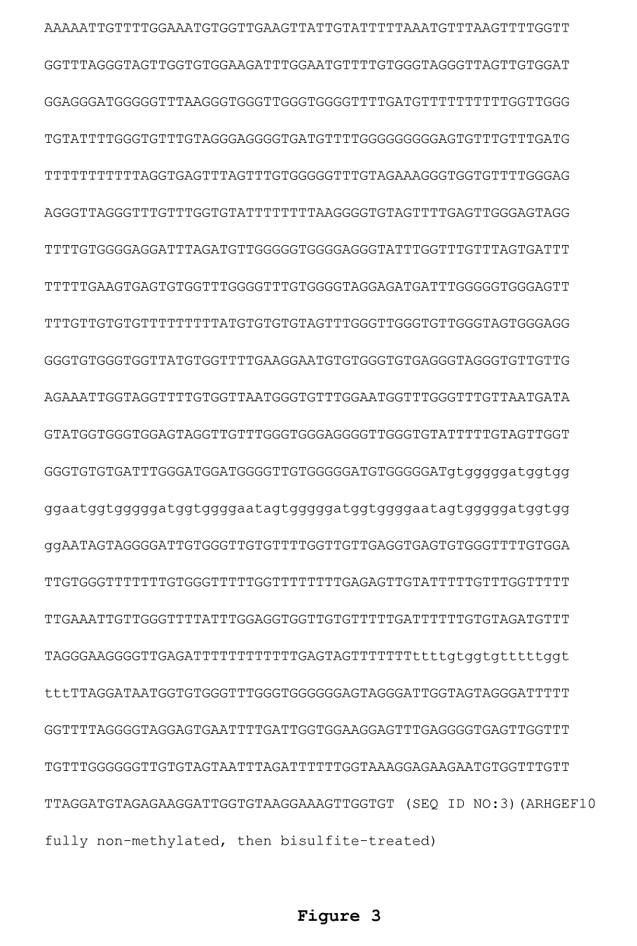

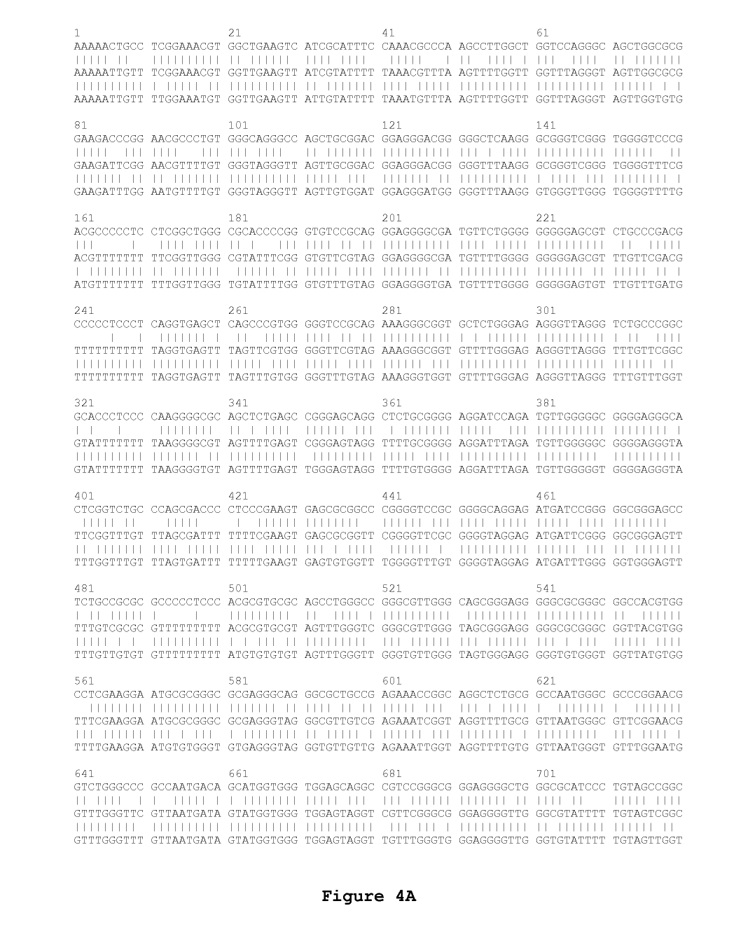

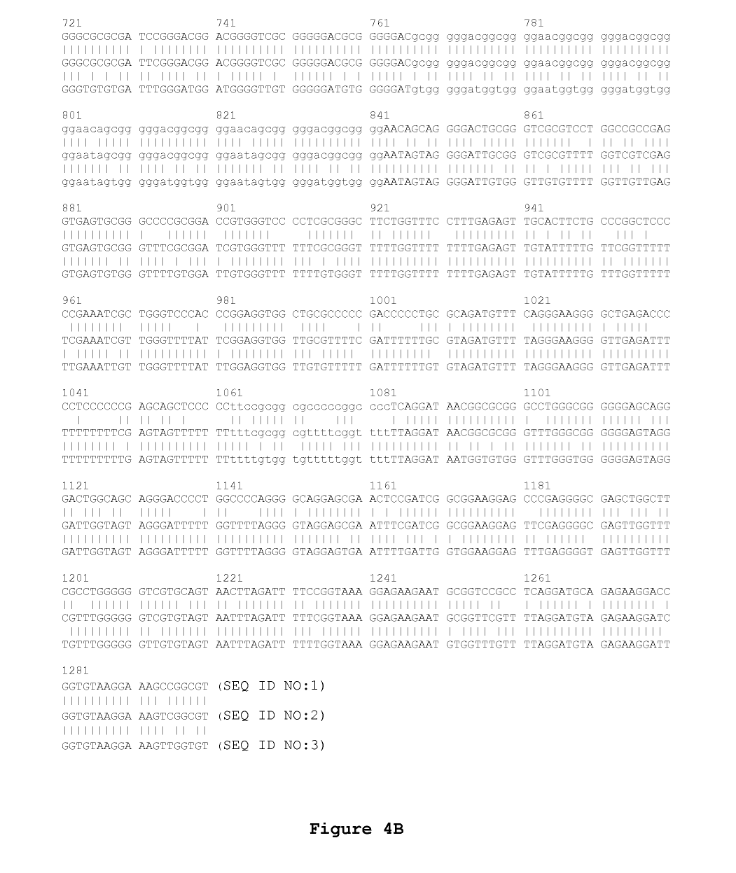

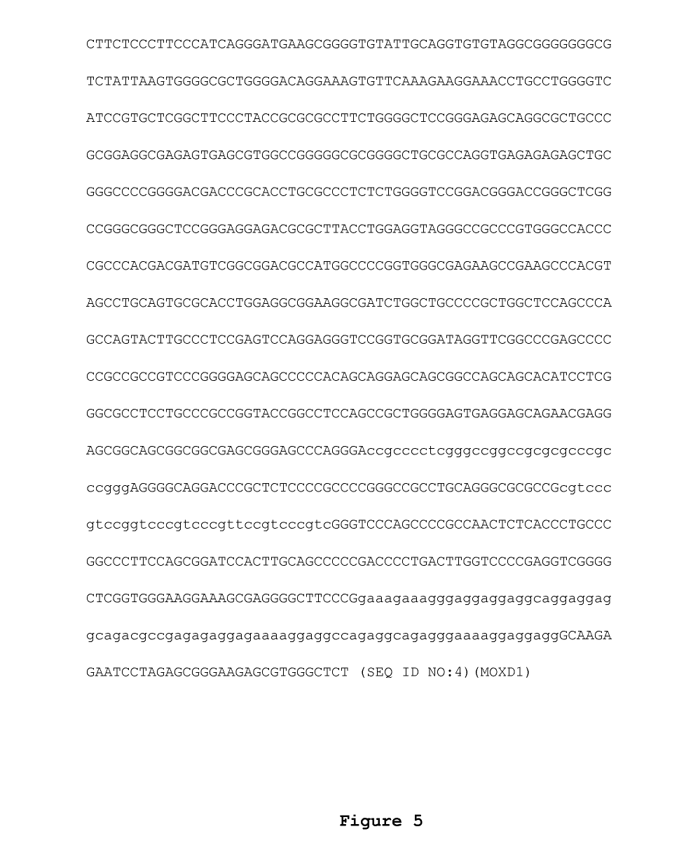

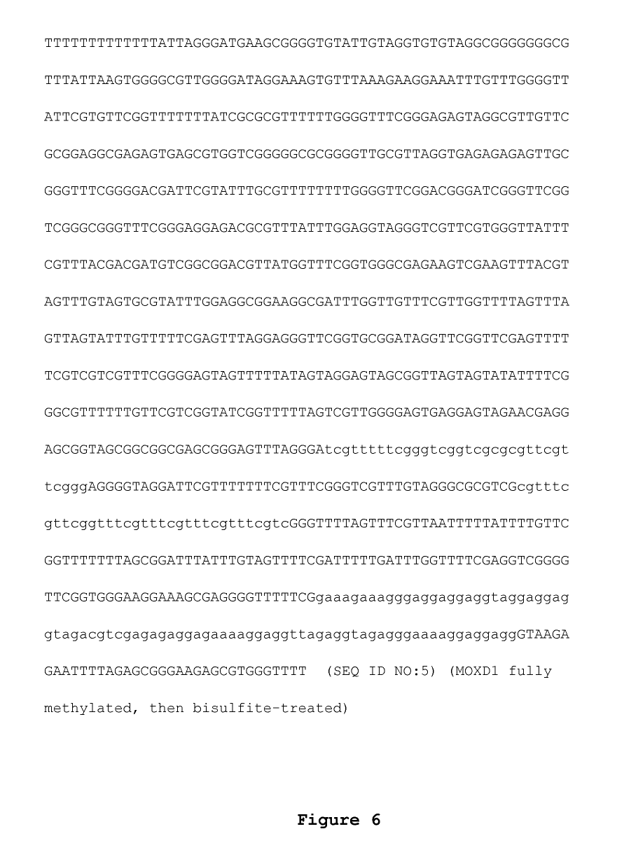

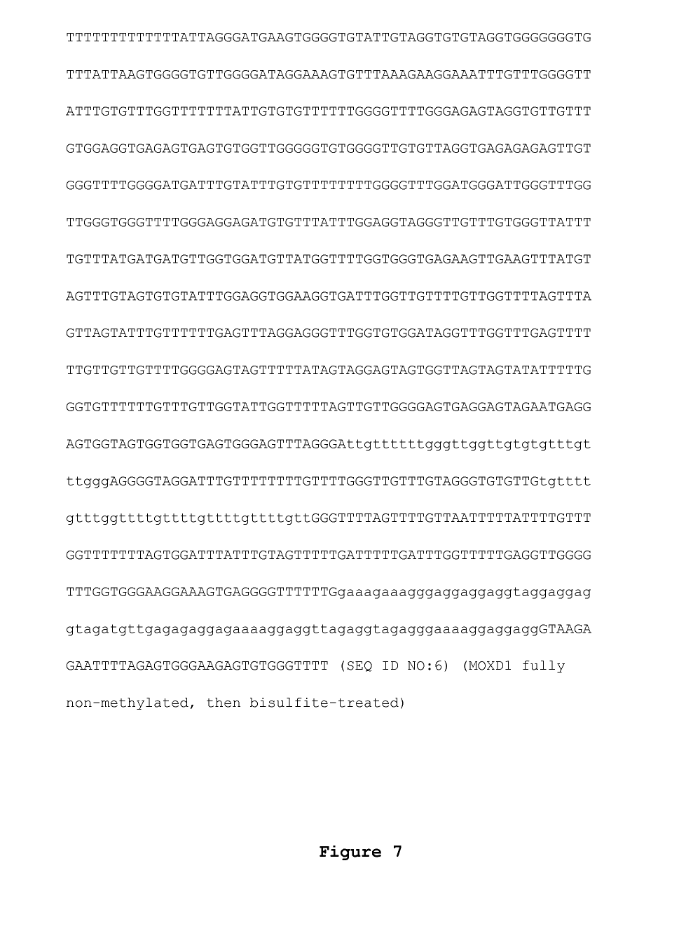

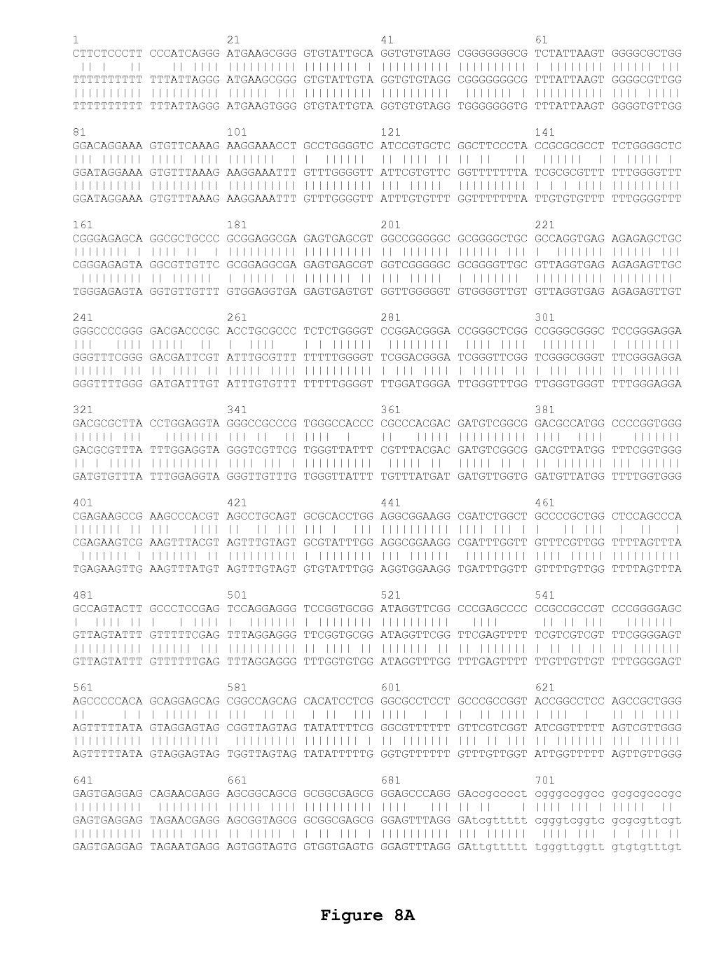

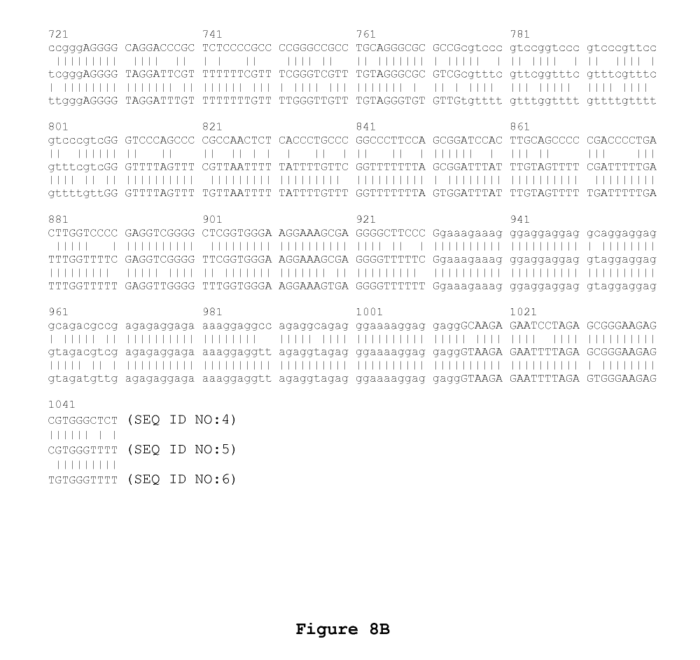

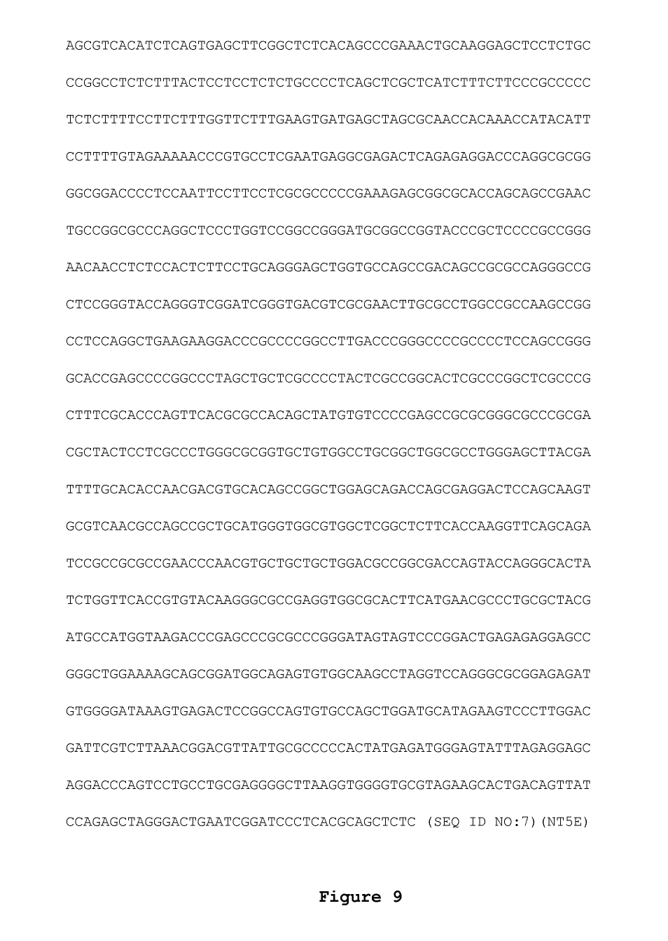

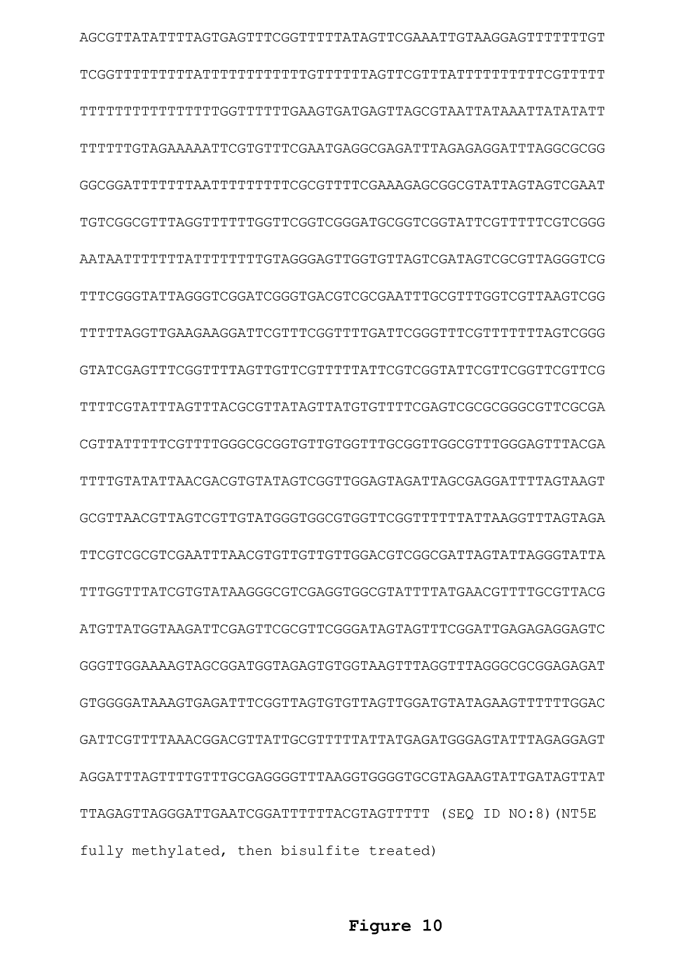

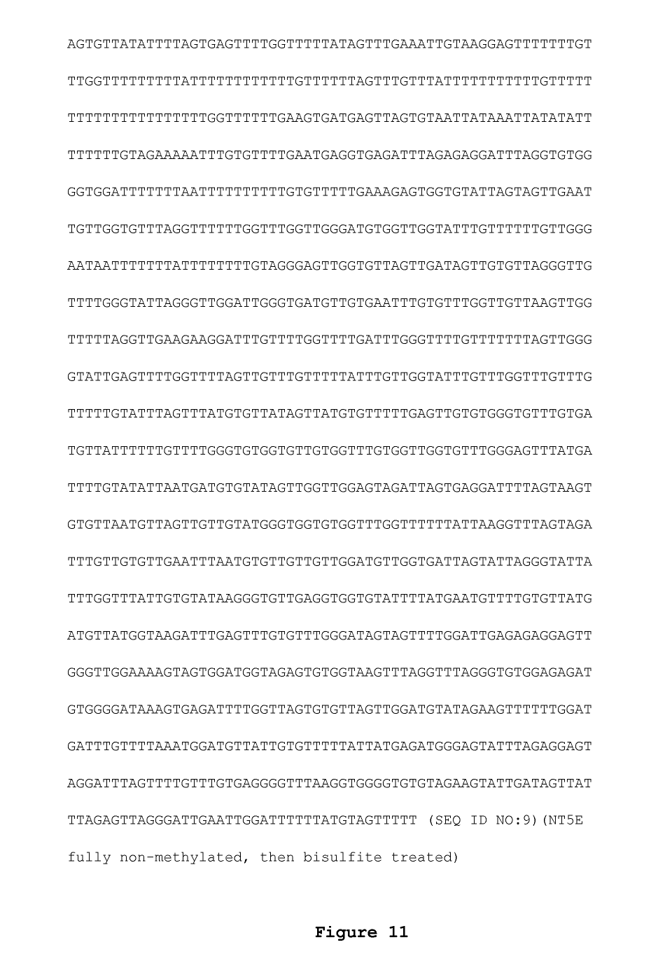

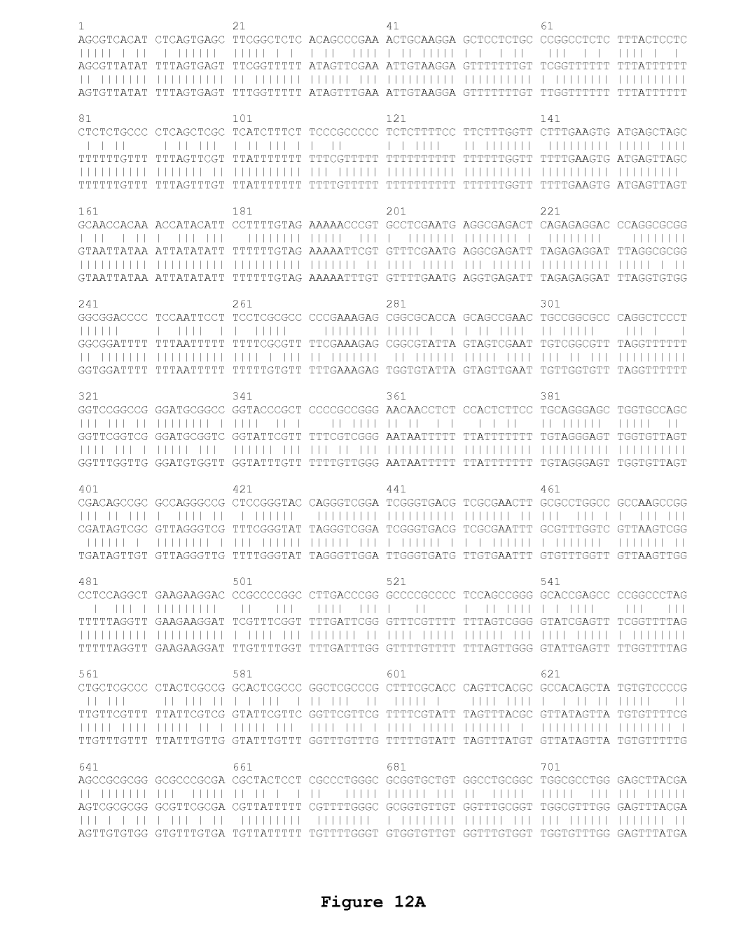

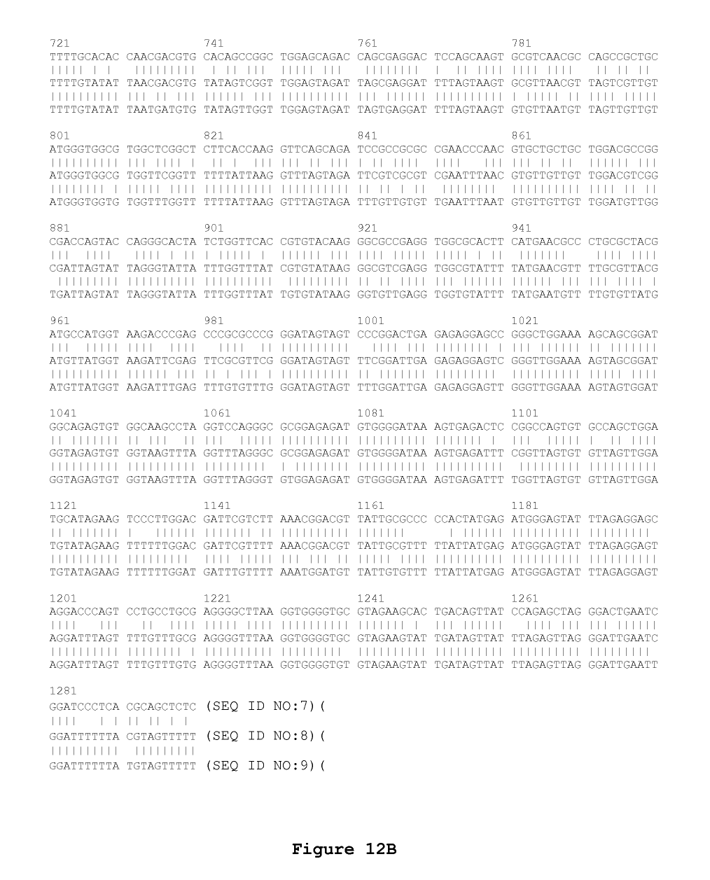

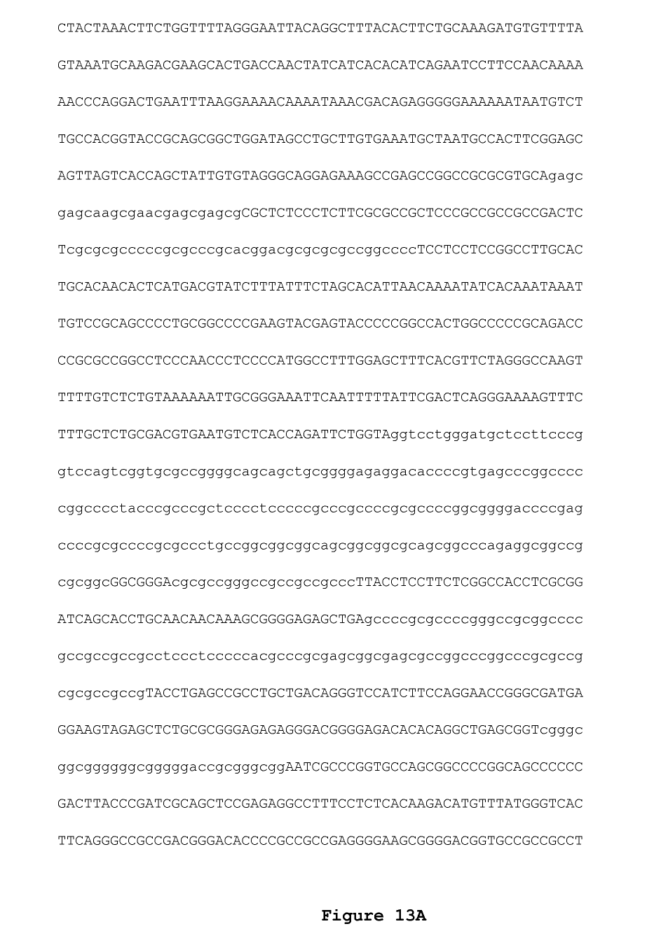

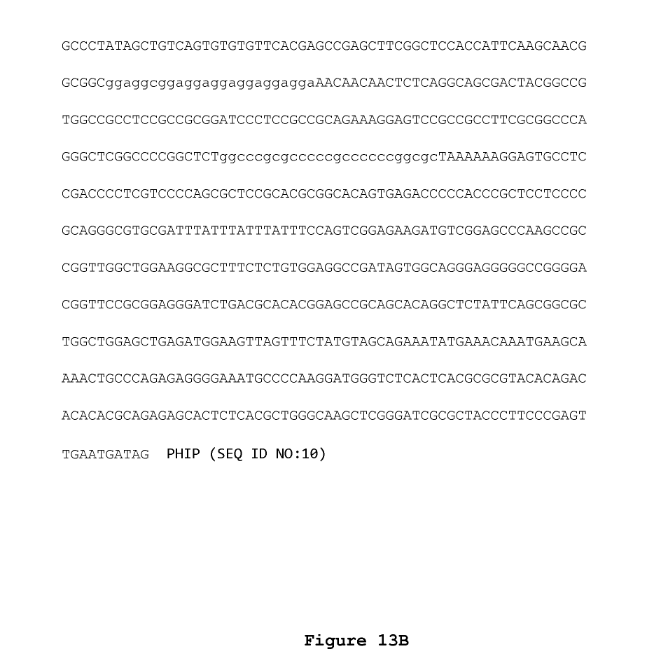

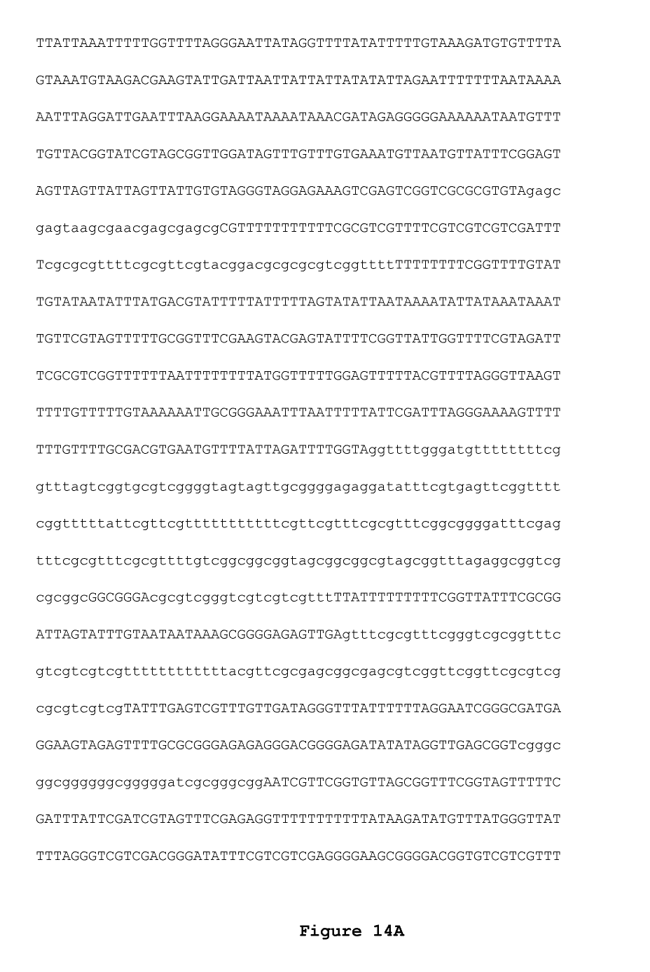

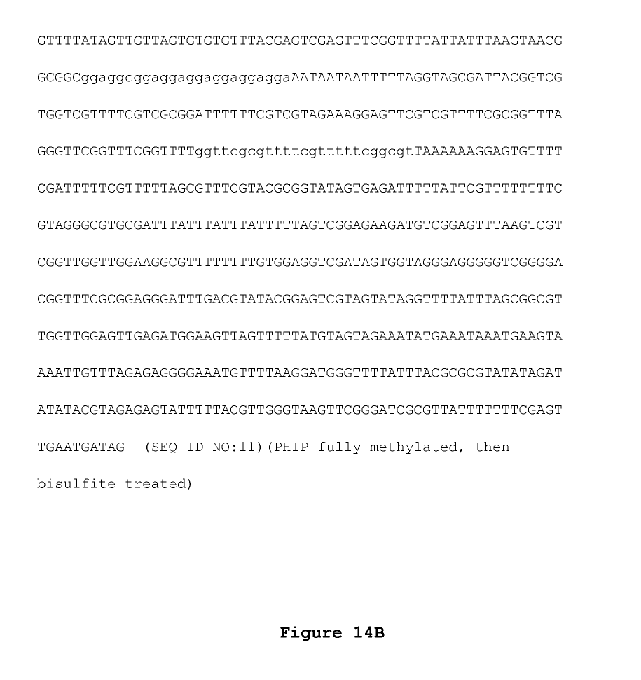

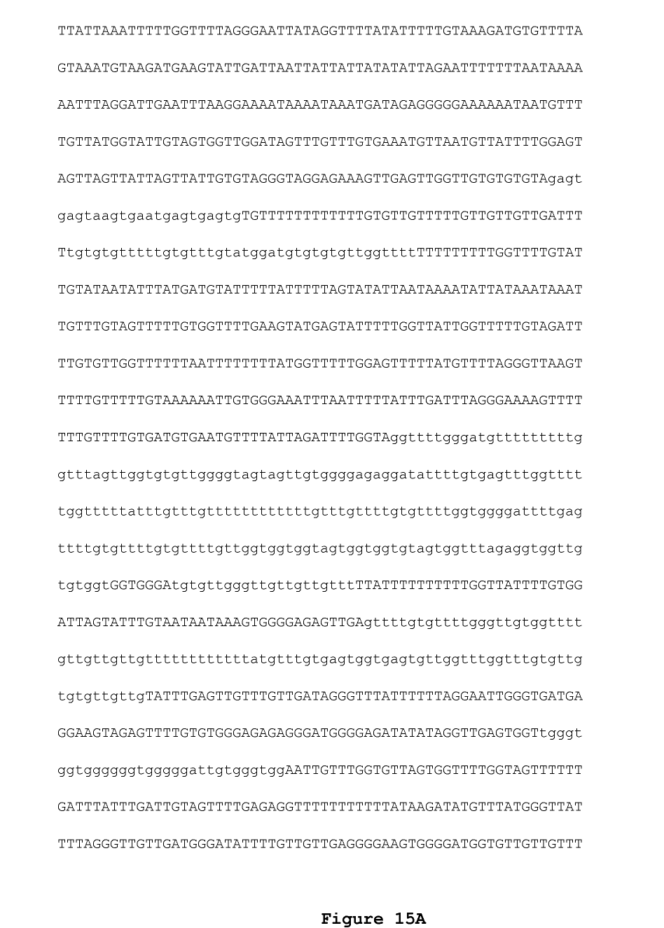

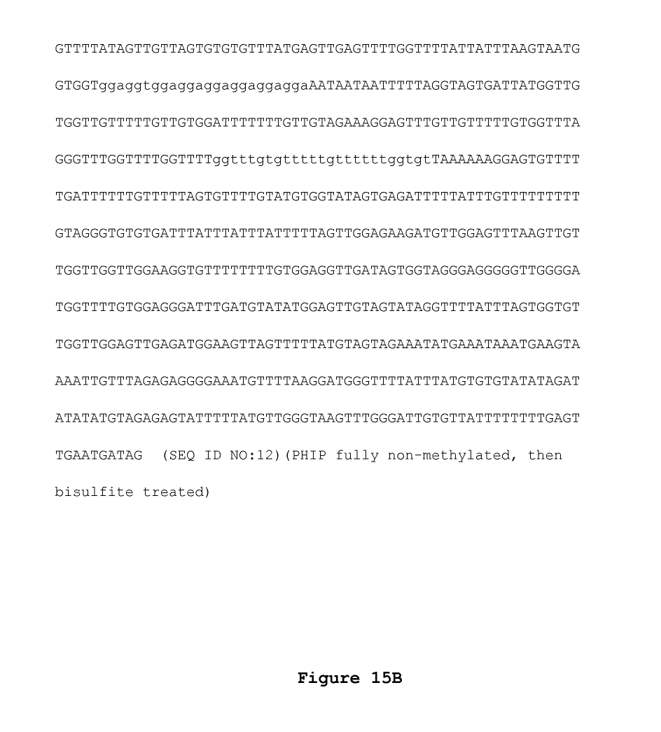

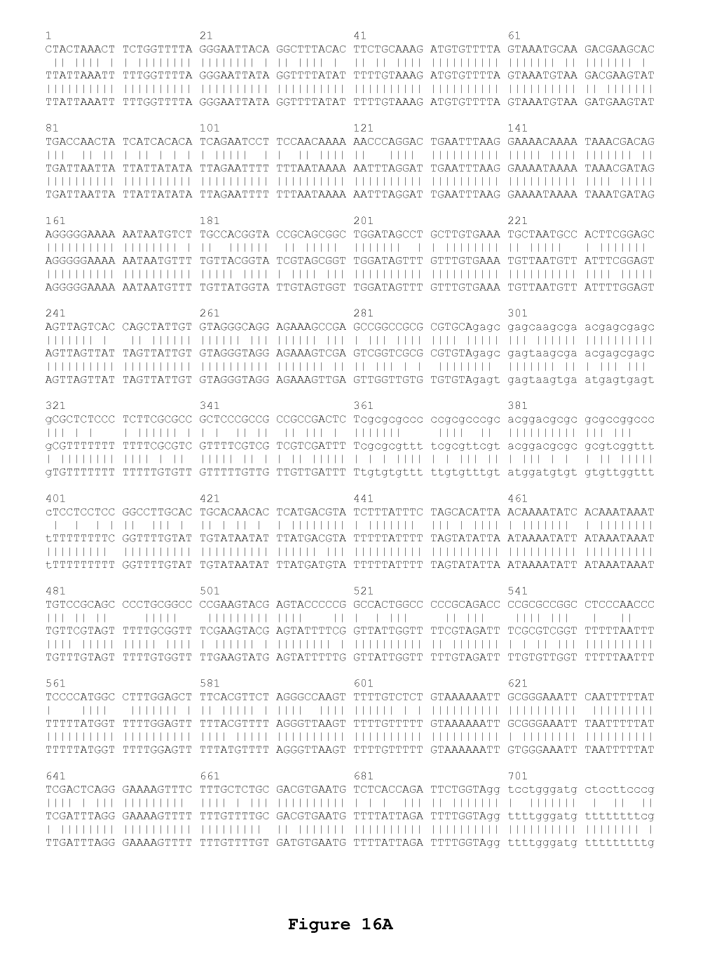

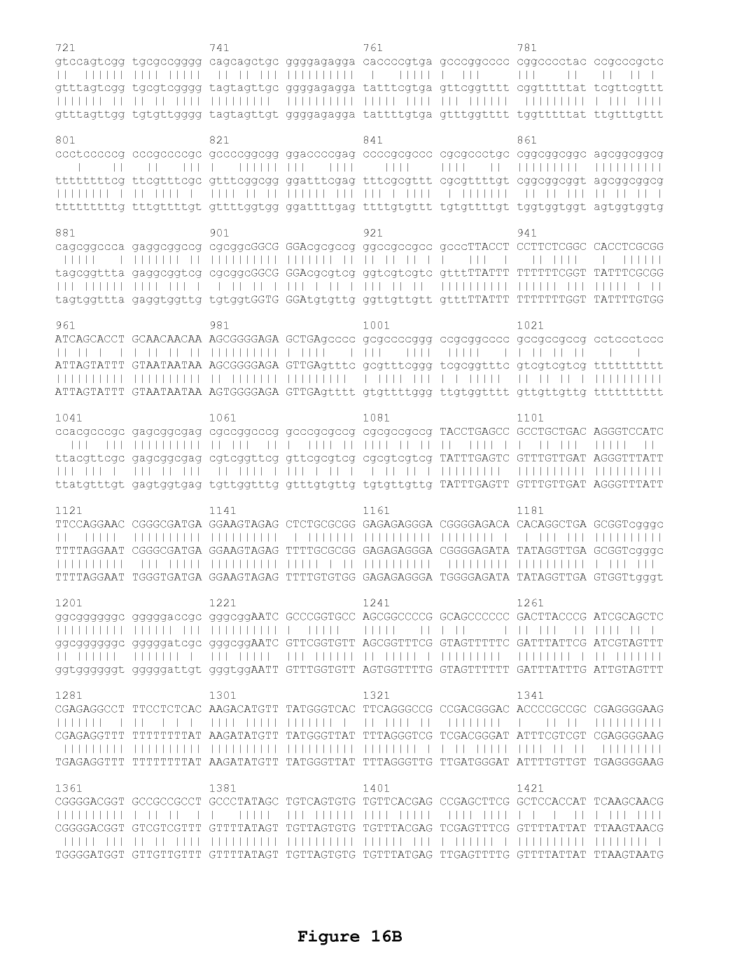

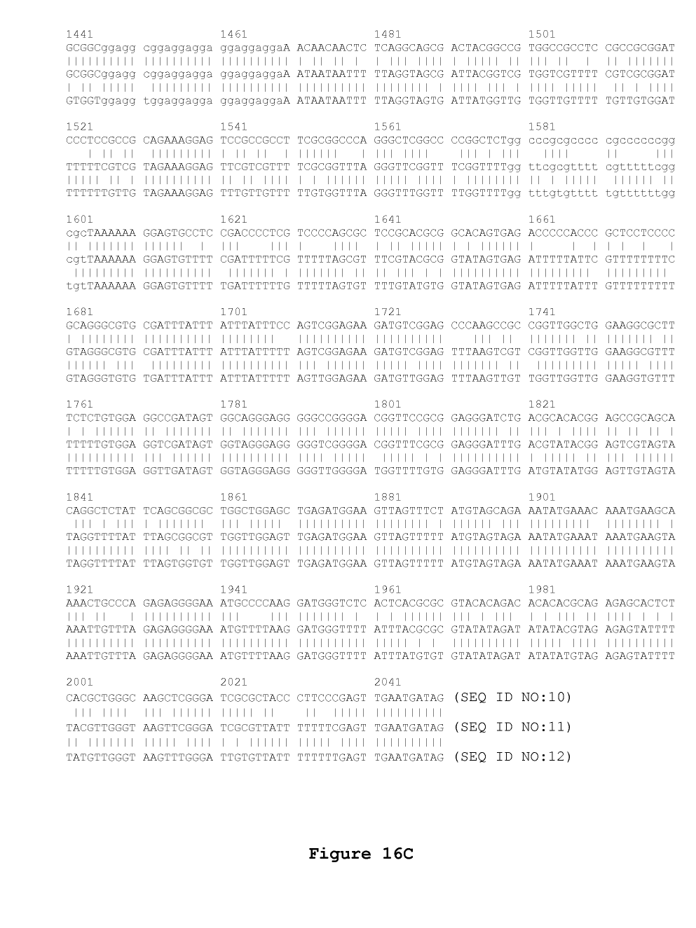

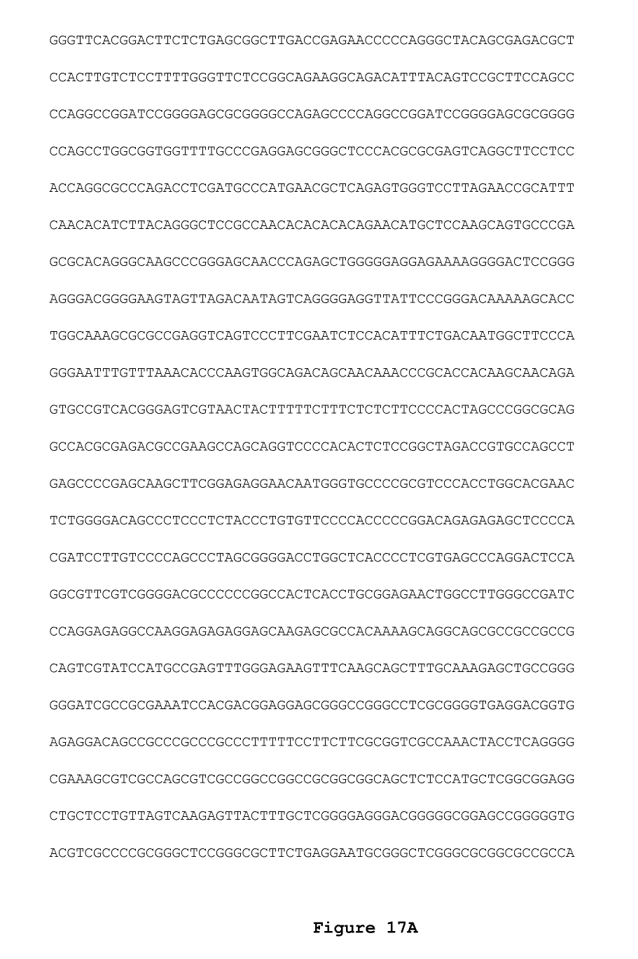

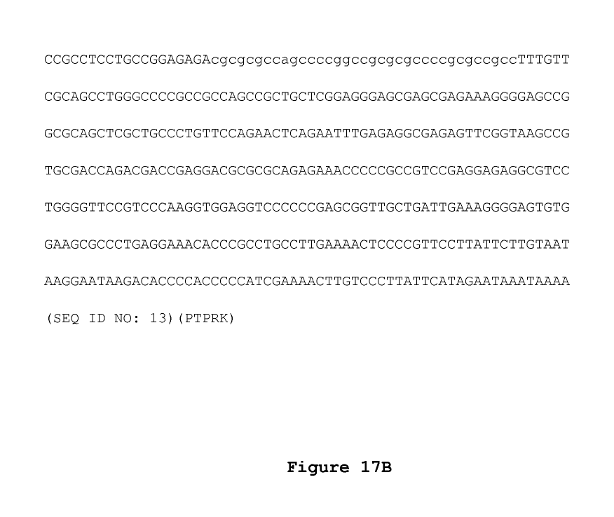

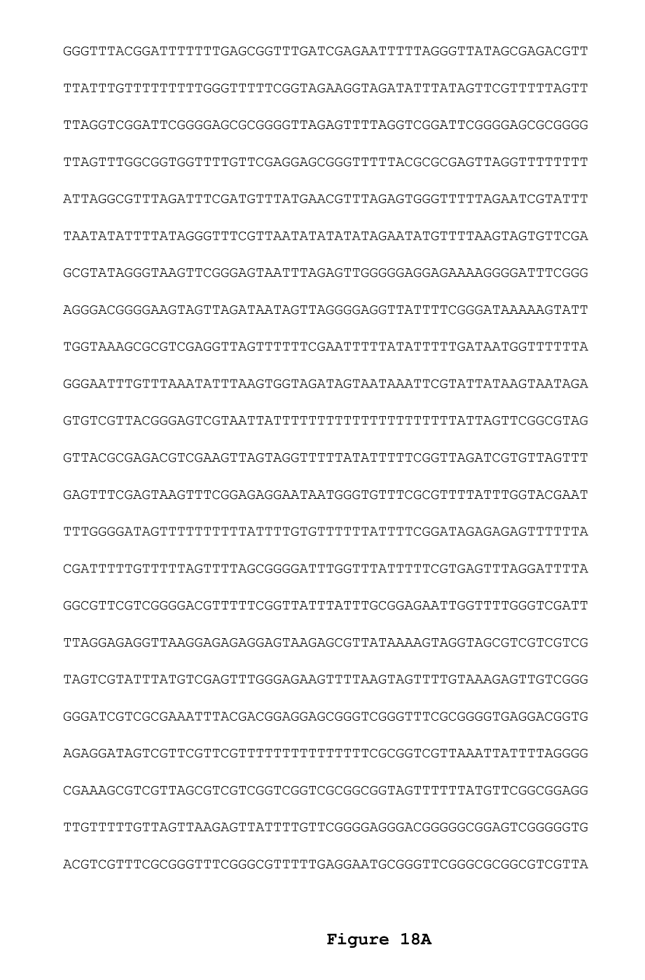

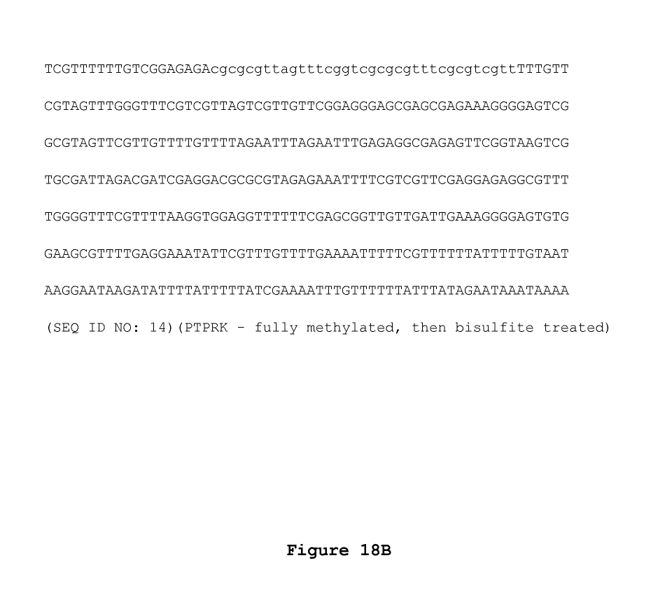

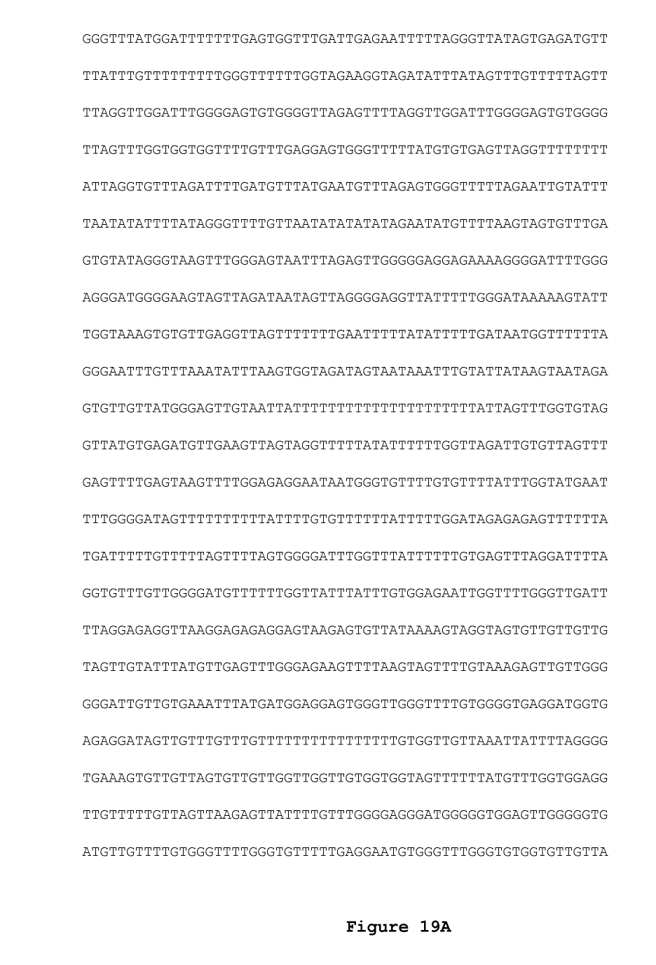

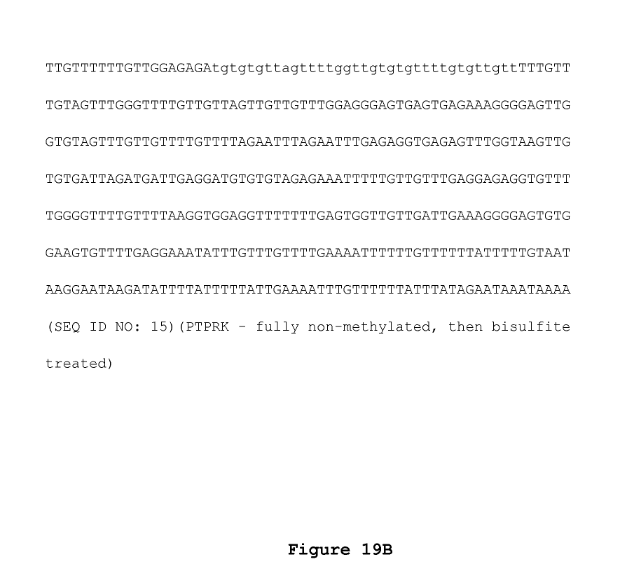

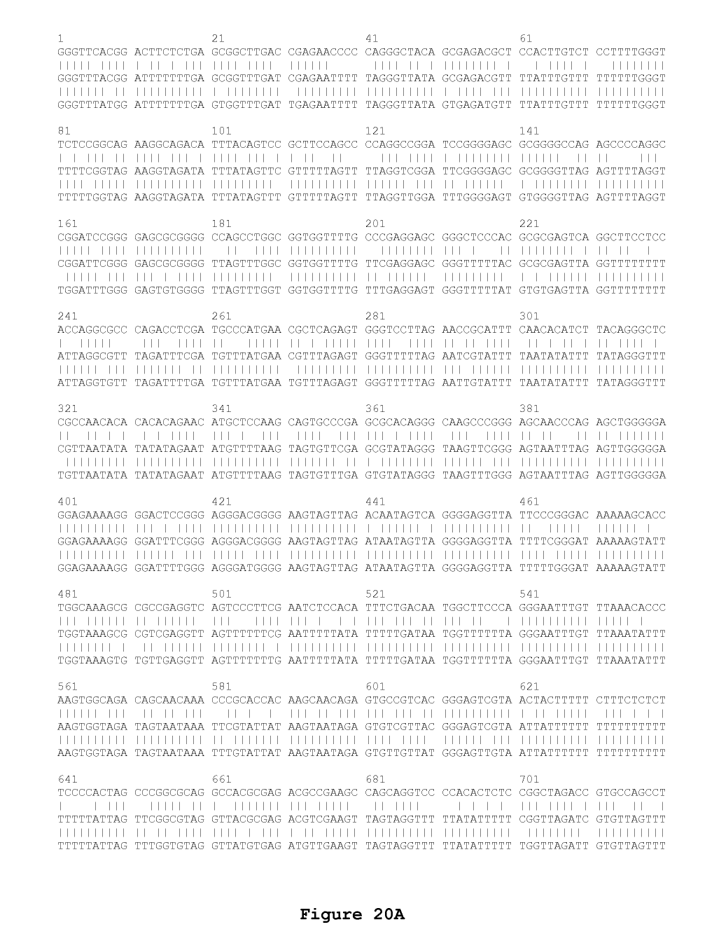

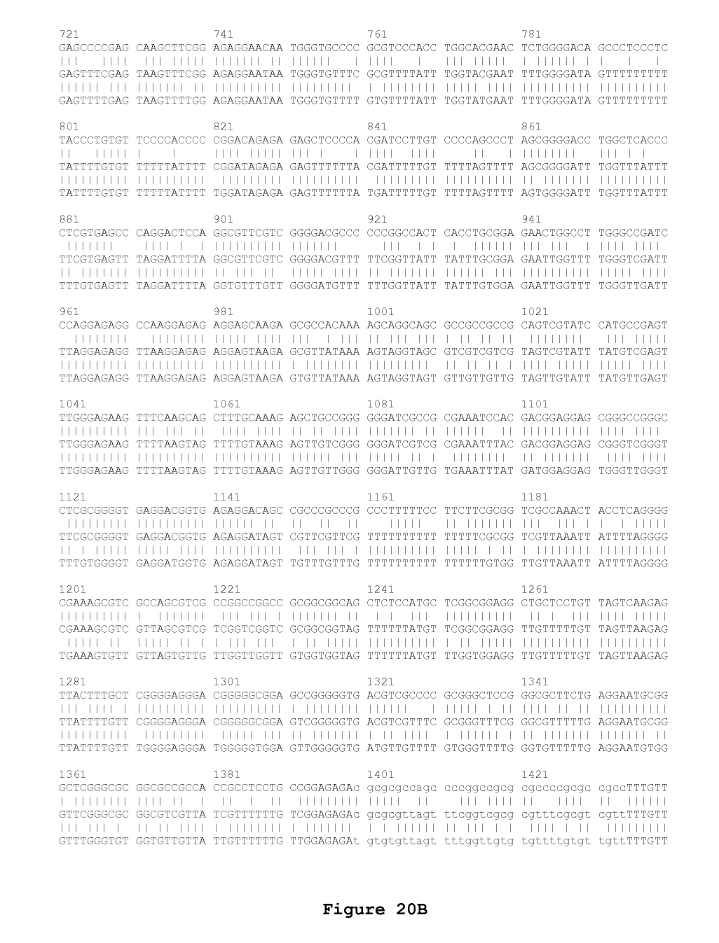

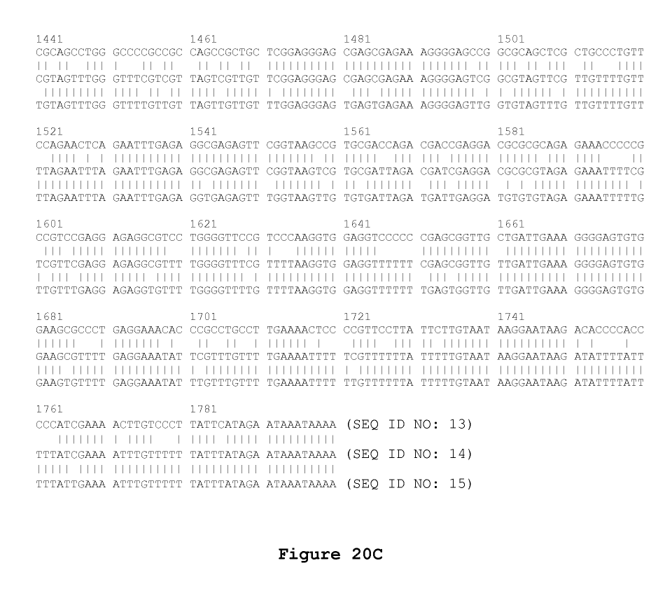

Sequences are presented in accordance with convention beginning at the 5′ terminus and proceeding from left to right and top to bottom.

### DETAILED DESCRIPTION OF THE INVENTION

The invention provides a method of diagnosing cancer by assaying for the methylation of one or more CpG islands that are indicative of cancer. Cancer can include, for example, breast, lung, liver, pancreas, head and neck, throat, thyroid, esophagus, brain, ovarian, kidney, skin, colorectal, and hematopoeietic (e.g., lymphomas and leukemic) cancer. Generally, the method comprises providing a biological sample from a subject in need of cancer diagnosis and assaying the sample for methylation of one or more CpG islands associated with at least one gene selected from the group consisting of: ARHGEF10, NT5E, MOXD1, PTPRK, and PHIP. In preferred embodiments, the method can include assaying for methylation of CpG islands associated with two, three, four, or five of the foregoing genes. Methylation of the CpG islands associated with these genes is indicative of cancer.

The invention further provides a method of diagnosing prostate cancer by assaying for the methylation of one or more CpG islands that are indicative of prostate cancer in a male mammal, e.g., a human or dog. In one embodiment, the method comprises providing a biological sample from a male mammal in need of cancer diagnosis, e.g., a male human undergoing prostate cancer evaluation, and assaying the sample for methylation of one or more CpG islands associated with at least one gene selected from the group of: ARHGEF10, NT5E, MOXD1, PTPRK, and PHIP. In preferred embodiments, the method of diagnosing prostate cancer can include assaying for methylation of CpG islands associated with two, three, four, or five of the foregoing genes. For example, the method of diagnosing prostate cancer includes assaying the biological sample for methylation of one or more CpG islands associated with one, two, or three genes selected from the group of: NT5E, MOXD1, and PTPRK. Methylation of the CpG islands associated with these genes is indicative of cancer.

The foregoing method of diagnosing prostate cancer can optionally include, in combination with assaying for methylation of CpG islands associated with the foregoing genes, further assaying the biological sample for methylation of one or more CpG islands associated with at least one gene that is known to be (i) methylated in prostate cancer and (ii) not detectably methylated or methylated at a lower level (e.g., about 50% or less, about 40% or less, about 30% or less, about 20% or less, or less than about 10%) in BPH. In this regard, when the method includes assaying for at least one CpG island that is known to be methylated in prostate cancer but is known not to be detectably methylated or methylated at a lower level in BPH, the method preferably includes assaying the biological sample for methylation of CpG islands associated with at least three different genes. Examples of CpG islands known to be methylated in prostate cancer but not detectably methylated or methylated at a lower level in BPH include CpG islands disclosed in U.S. Patent Application Publication 2011/0097728 A1, which published Apr. 28, 2011 and is incorporated herein by reference in its entirety, e.g., neuregulin cell-surface ligand (NRG1), kinesin family member 13B (KIF13B), neurogenin 3 transcription factor (NEUROG3), paladin (predicted protein tyrosine phosphatase) (PALD), methyltransferase family member 1 (HEMK1), fibroblast growth factor receptor 20 (FGF20), nodal homolog (TGF-β signaling pathway) (NODAL), Kinesin family member C2 (KIFC2), Glutathione peroxidase 7 (GPX7), Ras association (RalGDS/AF-6) domain family 5 (RASSF5). Additional examples of CpG islands known to be methylated in prostate cancer but not detectably methylated or methylated at a lower level in BPH include CpG islands associated with glutathione S-transferase P1 (GSTP1), adenomatosis polyposis coli (APC), Cub and Sushi multiple domains1 (CSMD1), tumor necrosis factor receptor superfamily member 10A (TNFRSF10A) tumor necrosis factor receptor superfamily member 10B (TNFRSF10B), tumor necrosis factor receptor superfamily member 10C (TNFRSF10C), tumor necrosis factor receptor superfamily 10D (TNFRSF10D), secreted frizzled-related protein 1 (SFRP1), secreted frizzled-related protein 2 (SFRP2), dickkopf homolog 3 (DKK3), prostaglandin-endoperoxide synthase 2 (PTGS2), cyclin-dependent kinase inhibitor 1C(CDKN1C/p57), Ras association (RalGDS/AF-6) domain family 1 (RASSF1), and G-protein coupled receptor 62 (GPR62).

The invention further provides a method of treating cancer, e.g., prostate cancer, based on the methods of diagnosis disclosed herein. For example, when the biological sample includes one or more methylated CpG islands associated with at least one gene selected from the group of: ARHGEF10, NT5E, MOXD1, PTPRK, and PHIP (and, optionally, the presence of one or more CpG islands known to be methylated in prostate cancer but not detectably methylated or methylated at a lower level in BPH), the subject can be treated for prostate cancer.

Methods of treating prostate cancer can include radiation therapy, hormone therapy and/or surgical removal of prostate tissue. Radiation therapy can be delivered from an external radiation source or from an internal source (e.g., by radioactive particles or brachytherapy). Hormone therapy includes the administration of drugs that block the production of testosterone, the administration of drugs that prevent testosterone from interacting with prostate cancer cells, or removal by surgery of testosterone producing tissue (orchiectomy). The surgical removal of prostate tissue includes radical prostatectomy by retropubic, perineal, laparascopic, or robotic surgery. Other methods of treating prostate cancer include cryosurgery or cryoablation, high intensity focused ultrasound heating, and chemotherapy.

The invention also provides a method of prognosticating cancer by assaying for the methylation of one or more genes that are indicative of the grade or stage of the cancer, and/or the length of disease-free survival following treatment for cancer. Generally, the method comprises providing a biological sample from a subject in need of cancer prognosis and assaying the sample for methylation of one or more CpG islands associated with at least one gene selected from the group consisting of: ARHGEF10, NT5E, MOXD1, PTPRK, and PHIP. In preferred embodiments, the method can include assaying for methylation of CpG islands associated with two, three, four, or five of the foregoing genes. Methylation of the CpG islands associated with these genes is indicative of the grade or stage of the cancer, and/or the length of disease-free survival following treatment for cancer.

The invention also provides a method of prognosticating prostate cancer in a male mammal by assaying for the methylation of one or more CpG islands that are indicative of the grade or stage of the prostate cancer, and/or the length of disease-free survival following treatment for prostate cancer. In one embodiment, the method comprises assaying a biological sample from the male mammal for methylation of one or more CpG islands associated with at least one of the following genes: ARHGEF10, NT5E, MOXD1, PTPRK, and PHIP. In preferred embodiments, the method of diagnosing prostate can include assaying for methylation of CpG islands associated with two, three, four, or five of the foregoing genes. For example, the method of diagnosing prostate cancer includes assaying the biological sample for methylation of CpG islands associated with NT5E, MOXD1, and PTPRK. Methylation of the CpG islands associated with these genes is indicative of the grade or stage of prostate cancer, and/or the length of disease-free survival following treatment for prostate cancer.

The foregoing method of prognosticating prostate cancer can optionally include, in combination with assaying for methylation of CpG islands associated with the foregoing genes, further assaying the biological sample for methylation of one or more CpG islands associated with at least one gene that is known to be (i) methylated in prostate cancer and (ii) not detectably methylated or methylated at a lower level (e.g., about 50% or less, about 40% or less, about 30% or less, about 20% or less, or less than about 10%) in BPH. Percent methylation level in BPH refers to the percent of patients that exhibit some detectable level of methylation at that locus. In this regard, when the method includes assaying for methylation of at least one CpG island that is known to be methylated in prostate cancer but is known not to be detectably methylated or is methylated at a lower level in BPH, the method preferably includes assaying the biological sample for methylation of CpG islands associated with at least three different genes. Examples of CpG islands known to be methylated in prostate cancer but not detectably methylated or methylated at a lower level in BPH include CpG islands associated with NRG1, KIF13B, NEUROG3, PALD, HEMK1, FGF20, NODAL, KIFC2, GPX7, RASSF5, GSTP1, APC, CSMD1, TNFRSF10A, TNFRSF10B, TNFRSF10C, TNFRSF10D, SFRP1, SFRP2, DKK3, PTGS2, CDKN1C/p57, RASSF1, and GPR62. Methylation of CpG islands associated with the genes is indicative of the grade or stage of the prostate cancer, and/or the length of disease-free survival following treatment for prostate cancer.

Obtaining information about the aggressiveness of the cancer, its grade, and its stage is helpful when choosing a course of treatment. The patterns of CpG methylation may be correlated to the pathological stage and grade of the tumor. For example, in prostate cancer, patterns of CpG methylation may be correlated to the Gleason score of the primary tumor. The molecular information derived from CpG methylation may also be correlated to the likelihood of survival and the length of disease-free survival following treatment. The above prognostic methods can enable the prediction of the course of the cancer, as well as the prediction of the best approach to treatment.

Also provided are methods of assessing the efficacy of treatment of cancer by assaying for the reduced methylation of CpG islands, which indicates efficacy of treatment. Generally, the method comprises providing a first and a second biological sample from a subject in need of assessing the efficacy of treatment of cancer and assaying the samples for a change in methylation level of a CpG island associated with at least one gene selected from the group consisting of: ARHGEF10, NT5E, MOXD1, PTPRK, and PHIP. Generally, the first biological sample is taken (e.g., prior to commencing treatment or during treatment) before the second biological sample, and the second biological sample is taken after a course of treatment. In preferred embodiments, the method includes assaying for a change in methylation of CpG islands associated with two, three, four, or five of the foregoing genes. For example, the method of diagnosing prostate cancer includes assaying the biological sample for methylation of CpG islands associated with one, two, or all three of NT5E, MOXD1, and PTPRK. A decrease or absence of methylation of the assayed one or more CpG islands in the second sample (i.e., following the course of treatment) indicates that the treatment is effective. Alternatively, the maintenance or increase of methylation in the assayed CpG islands in the second sample can indicate a reduction or absence of treatment efficacy.

The invention provides a method of assessing the efficacy of treatment of prostate cancer in a male mammal by assaying for the reduced methylation of CpG islands, which indicates efficacy of treatment of prostate cancer. In one embodiment, the method comprises assaying biological samples, which are taken from the male human periodically during the course of treatment, for methylation of one or more CpG islands associated with at least one gene selected from the group consisting of: ARHGEF10, NT5E, MOXD1, PTPRK, and PHIP. In preferred embodiments, the method can include assaying for methylation of CpG islands associated with two, three, four, or five of the foregoing genes. For example, the method of assessing the efficacy of treatment of prostate cancer includes assaying the biological sample for methylation of CpG islands associated with one, two, or all three of NT5E, MOXD1, and PTPRK. Generally, the assayed biological samples in the method include a first and a second biological sample. The first biological sample can be taken, for example, prior to commencing treatment or during treatment, though in any event prior to taking the second biological sample. The second biological sample is taken during or after a course of treatment. A decrease or absence of methylation of the assayed one or more CpG islands in the second sample (i.e., following the course of treatment) as compared to the first sample indicates that the treatment is effective. Alternatively, the maintenance or increase of methylation in the assayed CpG islands in the second sample as compared to the first sample can indicate a reduction in or absence of treatment efficacy.

The foregoing method of assessing the efficacy of prostate cancer treatment can optionally include, in combination with assaying for methylation of CpG islands associated with the foregoing genes, further assaying the biological sample for reduced methylation of one or more CpG islands associated with at least one gene that is known to be (i) methylated in prostate cancer and (ii) not detectably methylated or methylated at a lower level (e.g., about 50% or less, about 40% or less, about 30% or less, about 20% or less, or less than about 10%) in BPH. In this regard, when the method includes assaying the biological samples for methylation of at least one CpG island that is known to be methylated in prostate cancer but known not to be detectably methylated or methylated at a lower level in BPH, the method preferably includes assaying for methylation of CpG islands associated with at least three different genes. Examples of CpG islands known not to be methylated in prostate cancer but not detectably methylated or methylated at a lower level in BPH include NRG1, KIF13B, NEUROG3, PALD, HEMK1, FGF20, NODAL, KIFC2, GPX7, RASSF5, GSTP1, APC, CSMD1, TNFRSF10A, TNFRSF10B, TNFRSF10C, TNFRSF10D, SFRP1, SFRP2, DKK3, PTGS2, CDKN1C/p57, RASSF1, and GPR62. A decrease or absence of methylation of the CpG islands associated with the assayed genes in the second sample as compared to the first sample following some or the entire course of treatment indicates that the treatment is effective. Alternatively, the maintenance or increase of methylation in the assayed CpG islands in the second sample as compared to the first sample can indicate a reduction or absence of treatment efficacy.

The invention further provides a method of treating cancer based on the foregoing assessment of treatment efficacy. When the assessment indicates that treatment is efficacious for the subject, the method includes continuing the same course of prostate cancer treatment or foregoing additional treatment (e.g., when radical prostectomy is successful). Alternatively, when the foregoing assessment indicates that treatment is not efficacious for the subject, the method includes providing the subject with additional or different treatment for prostate cancer. Additional treatment for prostate cancer can include another surgical procedure of the same type that was previously done. Additional treatment can refer to an increased dose or more frequent dosing regimen of hormone therapy, radiation therapy, or chemotherapy. A different treatment refers to a change in therapeutic approach, i.e., using a therapeutic regimen to treat the subject's prostate cancer which differs from that previously used to treat the subject. A different treatment can include changing from non-surgical to surgical therapy, changing from one type of surgical therapy to a different type of surgical therapy, changing from surgical to non-surgical (e.g., hormone or radiation) therapy, or changing from one type of non-surgical therapy to a different type of non-surgical therapy for prostate cancer.

CpG islands (Bird, Nature, 321: 209-213 (1986); and Gardiner-Garden et al., J. Molec. Biol., 196: 261-282 (1987)) comprise about 1% of vertebrate genomes and account for about 15% of the total number of CpG dinucleotides. CpG islands typically are between about 0.2 and about 2.0 kb in length. They can be located upstream of (e.g., in a promoter or enhancer region) of the coding sequence of the associated genes or they may also extend into or be found within gene-coding regions of their associated genes. A gene-coding region can include exons and introns. Use of the phrase “associated with” to describe a CpG island's relation to a gene, is intended to encompass CpG islands that are upstream of gene coding sequences as well as internal CpG islands. Some CpG islands are associated with the promoter of two genes and it can affect the expression of both genes. CpGs were labeled based on their location with respect to the nearest gene. In some cases, a CpG island may be located near the promoter of two different genes and may in this case influence the expression of both genes. In such cases, the CpG island is named after one of the genes. A CpG island can also be associated with a pseudogene or be located in a genomic region that includes no known genes or pseudogenes. The CpG island can still be of interest so long as its methylation status correlates with a disease status.

A CpG island can be separated by up to 25 kilobases (kb) (e.g., up to 20 kb, up to 19 kb, up to 18, kb, up to 17 kb, up to 16 kb, up to 15 kb, up to 10 kb, up to 9 kb, up to 8 kb, up to 7 kb, up to 6 kb, up to 5 kb, up to 4 kb, up to 3 kb, up to 2 kb, or up to 1 kb) from the transcription start site for the nearest gene and still be considered “associated with” the gene. Preferably, CpG islands associated with at least three genes are assayed. However, CpG islands associated with 4, 5, 6, 7, 8, 9, 10, 11, 12, 13, 14, 15, 16, 17, 18, 19, 20, 21, 22, 23, 24, 25, 26, 27, or even more genes can be assayed.

Methods of identifying CpG islands have been described (e.g., Takai et al., Proc. Nat'l. Assoc. Sci. USA, 99:3740-3745 (2002)). For example, genomic sequences can be analyzed to identify segments containing CpG islands that are at least 200 bp in length, have at least a 60% GC content, and contain at least 7% CpG dinucleotides. Preferred sequences are at least 250 bp in length, are at least 60% GC rich, and contain at least 7% CpG dinucleotides. Moreover, undesirable highly repetitive sequences can be screened out using a repeat masker that filters out sequences. Desirable sequences contain less than 50% repeats (i.e., a sequence of reduced complexity or a sequence that is present at multiple genomic locations) within the length of the identified CpG island. Preferably, the CpG island is no more than 45%, 40%, 35%, 30%, 25%, 20%, 19%, 18%, 17%, 16%, 15%, 14%, 13%, 12%, or 11% repetitive. Most desirable sequences are no more than 10% repetitive. Examples of repetitive sequences are available, for example, at the web site for National Center for Biotechnology Information (NCBI).

“Biological sample” is intended to encompass any suitable sample that enables accurate assay of CpG island methylation. Suitable biological samples include, for example, prostate tissue samples. Other examples of suitable biological samples include, but are not limited to, bodily fluids and tissues, e.g., whole blood, blood plasma, blood serum, urine, saliva, needle aspirate, and cells (such as cells obtained from blood, such as epithelial cells). Such samples are obtained in accordance with methods known in the art. When the biological sample is whole blood, blood plasma, or urine, preferably, CpG islands associated with more than three genes are assayed.

A CpG island is “not detectably methylated” when it is not methylated or it is methylated at a level below the level of sensitivity of the assay method employed.

“Noncancerous” tissue can be benign or normal. Alternatively, but not preferably, the tissue can be diseased, as long as it is not cancerous.

A “subject” herein can refer to a male mammal, preferably, a male human. The subject can be in need of evaluation for cancer and, therefore, undergoing evaluation for cancer, e.g., prostate cancer. For example, the subject can be a male human previously diagnosed with and/or treated for prostate cancer. In some embodiments, the subject can be one that is undergoing treatment for prostate cancer.

Methods of assaying for methylation of CpG islands are known in the art and include, for example, restriction enzyme-based technology, such as one that employs digestion with a methylation-sensitive restriction endonuclease coupled with Southern blot analysis, methylation-sensitive enzymes and polymerase chain reaction (PCR), such as methylation-sensitive arbitrarily primed PCR (AP-PCR; see, e.g., Gonzalgo et al., Cancer Res., 57: 594-599 (1997)), restriction landmark genomic scanning (RLGS; see, e.g., Plass et al., Genomic 58: 254-262 (1999)), methylated CpG island amplification (MCA; see, e.g., Toyota et al., Cancer Res., 59: 2307-2312 (1999)), differential methylation hybridization (DMH; see, e.g., Huang et al., Human Mol. Genet., 8: 459-470 (1999)), and Not I-based differential methylation hybridization (see, e.g., International Patent Publication No. WO 02/086163). Other methods are described in U.S. Pat. App. Pub. No. 2003/0170684 and International Patent Publication No. WO 04/05122.

Another method of assaying methylation of CpG islands includes the use of methylation-dependent restriction endonucleases, which preferentially digest methylated DNA. The following are examples of methylation-dependent restriction endonucleases (their respective recognition sites with methylated nucleotides in parentheses) include: FspEI (CC(N)₁₂), McrBC (PuC(N₄₀₋₃₀₀₀)uC), MspJI (CNNR(N)₉), LpnPI (CCDG(N)₁₀). These methylation-dependent restriction endonucleases are available from New England Biolabs (Ipswich, Mass.). Methylation-dependent restriction endonucleases such as MspJI, LpnPI, and FspEI generate short fragments of about 31-32 base pairs in length which can be sequenced using next generation sequencing (NGS) applications. See e.g., Mardis, Annu. Rev. Genomics Hum. Genet., 9: 387-402 (2008) and Matzker, Nature Rev. Genetics, 11, 31-46 (January 2010). Most CpG islands contain multiple methylation-dependent restriction endonucleases recognition sites. Therefore, digesting genomic DNA with such enzymes such as MspJI, LpnPI, and FspEI result in short (31-32 base pair) fragments and larger fragments of variable length that are dependent on the distance between two recognition sites. Alternatively or additionally, the larger variable length fragments can be assayed by PCR, Q-PCR, or other methods.

Still another method of assaying for methylation of CpG islands can include cytosine conversion-based technology. Such technology relies on methylation status-dependent chemical modification of CpG islands (i.e., deamination of unmethylated cytosines in CpG islands) within isolated genomic DNA or fragments thereof followed by DNA sequence analysis. Such methods employ reagents like hydrazine and bisulfite salts. Bisulfite treatment followed by alkaline hydrolysis is described by Olek et al., Nucl. Acids Res., 24: 5064-5066 (1996); and Frommer et al., PNAS USA, 89: 1827-1831 (1992). The use of methylation-sensitive primers to assay methylation of CpG islands in isolated genomic DNA is described by Herman et al., PNAS USA, 93: 9821-9826 (1996), and in U.S. Pat. Nos. 5,786,146 and 6,265,171. Bisulfite-treated DNA can be subsequently analyzed by conventional molecular techniques, such as PCR amplification, fluorescence-based, real-time PCR (see, e.g., Eads et al., Cancer Res., 59: 2302-2306 (1999); Heid et al., Genome Res., 6: 986-994 (1996); and U.S. Pat. No. 6,331,393), sequencing, oligonucleotide hybridization detection, and methylation-sensitive single nucleotide primer extension (Ms-SNuPE; see, e.g., Gonzalgo et al., Nucl. Acids Res., 25: 2529-2531 (1997); and U.S. Pat. No. 6,251,594).

A preferred method of assaying for methylation of one or more CpG islands includes isolating genomic DNA (and/or fragments thereof) from a biological sample, treating the DNA under deaminating conditions that convert unmethylated cytosines to uracil, and using the treated DNA as a template in a PCR reaction to amplify a target sequence that includes the CpG-island of interest, thereby producing an amplified sequence. Unmethylated cytosines in the target sequence, which are converted to uracils by the deaminating treatment, are amplified as thymines in the corresponding position of the amplified sequence. Since the sequence of the forward and the reverse strand of the CpG island lose their complimentarity after the deamination reaction, the methylation status of the CpG island can be determined by assaying one or both of the original strands by utilizing primers capable of annealing to the strand of interest.

The deamination reaction may not proceed to completion, which results in false positives. For example, deamination of DNA sequences using bisulfite salt is sensitive to the purity of the DNA, length of incubation, and the secondary structure of the denatured templates. Quantitative PCR methods can be used to assay for the efficiency of deamination. However, quantitative PCR methods are limited to assaying the conversion status within the sites where the primers and probes anneal to the template.

Quantitative PCR methods are also limited to assaying for the methylation of cytosines within the sites where the primers and probes anneal to the template. The primers and the probe only anneal efficiently to the templates that are fully converted and contain methylation at the appropriate cytosine nucleotides. Thus, they fail to provide methylation information for CpG dinucleotides that are not assayed for. The CpG islands may also be analyzed using direct sequencing following the deamination treatment. However, due to the heterogeneity of the methylation pattern within a CpG island and the presence of homopolymeric stretches within the sequence, direct sequencing of CpG islands can yield a sequencing pattern that is too noisy and complex for the available sequencing software.

Another method of assaying for methylation of one or more CpG islands the use of terminator-coupled linear amplification. Terminator-coupled linear amplification is described in U.S. Patent Application Publication 2011/0097728 A1, which is incorporated by reference herein in its entirety.

The levels of methylation or patterns of methylation at given CpG islands can be assayed as appropriate. The assay can employ the use of a reference standard when appropriate to enable the determination of abnormal methylation. A reference standard can be determined based on reference samples obtained from age-matched noncancerous classes of adjacent tissues, and with normal peripheral blood lymphocytes. When, for example, efficacy of treatment is being assessed, the assay results of biological samples taken over the course of treatment can be compared without the use of a reference standard.

When the DNA obtained from a biological sample is in limited quantities and is not sufficient for the analysis of multiple markers, the methods described herein can include amplifying the DNA from the sample. Amplification can be done using PCR amplification or isothermal amplification methods, for example, those described in U.S. Pat. Nos. 5,854,033; 6,124,120; 6,143,495; 6,210,884; 6,642,034; 6,280,949; 6,632,609; and 6,642,034; and U.S. Pat. App. Pub. Nos. 2003/0032024; 2003/0143536; 2003/0235849; 2004/0063144; and 2004/0265897, which are incorporated herein by reference in their entirety. Isothermal amplification can include rolling circle or strand displacement amplification. Methods that combine PCR and isothermal amplification have also been described (U.S. Pat. Nos. 6,777,187; and 6,828,098; and U.S. Pat. App. Pub. Nos. 2004/0209298; 2005/0032104; and 2006/0068394, each of which is incorporated herein by reference in its entirety). U.S. Pat. App. Pub. No. 2005/0202490, which is incorporated herein by reference in its entirety, describes the use of such methods in combination with methylation-sensitive restriction enzymes to study the methylation pattern of DNA. DNA amplification can also include methylation-coupled whole genomic amplification to generate the DNA needed, such as described in U.S. Pat. App. Pub. No. 2006/0257905, which is incorporated by reference herein in its entirety. The methylation-coupled whole genomic amplification can be especially advantageous when DNA is recovered from minute biological samples or from bodily fluids such as urine or plasma.

Skilled artisans will appreciate that the various amplification methods described herein, e.g., the PCR amplification, isothermal amplification, and terminator-coupled linear amplification method, can employ nucleotides, nucleotide analogues, nucleotide or nucleotide analogue derivatives, and/or combinations thereof.

If desired, mRNA and protein levels can be assayed, and alterations in their expression levels can be indicative of a change in the level of methylation or the patterns of methylation at given CpG islands. Such methods of assaying mRNA and protein levels are also within the skill in the art. For example, the mRNA assay methods described in U.S. Provisional Patent Application No. 60/705,964 filed on Aug. 5, 2005 and International Patent Publication No. WO 2007/019444, which are incorporated herein by reference in their entirety, can be used. Such methods are particularly useful if a degraded tissue sample is used as the biological sample. Alternatively, reverse transcription with gene-specific primers can be used to assay mRNA levels. Proteins levels can be assayed, for example, using antibody and staining techniques.

It is important to note that, even though aberrant methylation of a CpG island can affect expression of the associated gene, the methods described herein are not dependent on a biological role for the hypermethylation. A hypermethylated CpG island can be useful in the methods of the invention regardless of its effect on gene expression. Accordingly, the only requirement is that there be a correlation between the methylated state of a CpG island and the presence of cancer.

The invention further provides target sequences and corresponding primers or probes that are useful in the above methods. The target sequences provide the context for the selection of CpG islands to assay for methylation. If a given target sequence contains more than one CpG island, all or less than all of the CpG islands, even one CpG dinucleotide, can be assayed for methylation with respect to that particular target sequence. The target sequence can include sequence in genomic DNA that is isolated from a subject's tissue or sequence in amplified genomic DNA, i.e., isolated genomic DNA that has been amplified in a way that preserves the methylation pattern of the original isolated genomic DNA. In this regard, a target sequence can include a subject's original genomic sequence associated with ARHGEF10, NT5E, MOXD1, PTPRK, and PHIP. For example, a target sequence can include ARHGEF10 promoter sequence, which is exemplified by SEQ ID NO: 1, NT5E, NT5E promoter sequence, which is exemplified by SEQ ID NO: 7, MOXD1 promoter sequence, which is exemplified by SEQ ID NO: 4, PTPRK promoter sequence, which is exemplified by SEQ ID NO: 13, or PHIP promoter sequence, which is exemplified by SEQ ID NO: 10.

A target sequence can also include the foregoing genomic sequences after the DNA is treated to produce deaminated DNA. Deamination can be done using bisulfite treatment or by any other method. The sequence of deaminated DNA will vary depending upon the extent that the original genomic DNA sequence was methylated. Thus, a target sequence can include the sequence of fully methylated DNA after bisulfite treatment. For example, a target sequence can include fully methylated, deaminated DNA. These fully methylated and deaminated sequences are used for illustrative purposed and do not exclude the use of partially methylated and deaminated sequences in the methods of the invention.

A target sequence can include a genomic sequence that is partially methylated, such as in DNA obtained from a tumor, and then deaminated such that the target differs from the sequence listed above. Persons of skill in the art will appreciate that a target sequence that includes a partially methylated and deaminated CpG island will result in a population of DNA molecules that differ at one or more positions that correspond to the cytosine residues in one or more CpG dinucleotides. Thus, a target sequence can include a variety of partially methylated and deaminated sequences based on one or more of the following genomic sequences SEQ ID NOs: 2 or 3 [ARHGEF10], SEQ ID NOs: 5 or 6 [MOXD1], SEQ ID NOs: 8 or 9 [NT5E], SEQ ID NOs: 11 or 12 [PHIP], and SEQ ID NOs: 14 or 15 [PTPRK].

These targets can be used in combination with known targets (for example known CpG islands associated with GSTP1, APC, CSMD1, TNFRSF10A, TNFRSF10B, TNFRSF10C, TNFRSF10D, SFRP1, SFRP2, DKK3, PTGS2, CDKN1C/p57, RASSF1, and GPR62. For example, fully or partially methylated and (subsequently) deaminated sequences for some of these genes are provided in U.S. Patent Application Publication 2011/0097728 A1, which is incorporated by reference herein in its entirety. Such target sequences can be isolated or purified in accordance with methods known in the art.

Also provided are isolated, purified, or synthesized primers derived from and suitable for amplifying sequences internal to the above isolated or purified target sequences. The isolated, purified, or synthesized primers can be DNA, RNA, peptide nucleic acid (PNA), and the like. It will be understood by one of ordinary skill in the art, however, that one type of nucleic acid can be preferred over another, depending on the particular biological sample, the methodology employed in assaying CpG islands for methylation, and the ability of the particular type of nucleic acid to detect methylation. One or more (e.g., two, three four, four, five, six, seven, eight, nine ten or more) isolated pairs of primers can be provided. Optionally, primers are provided as part of a kit useful in the methods disclosed herein. For example, each pair of primers can consist essentially of SEQ ID NOs: 16 and 17, SEQ ID NOs: 18 and 19, SEQ ID NOs: 20 and 21 SEQ ID NOs: 22 and 23, SEQ ID NOs: 24 and 25, SEQ ID NOs: 26 and 27, SEQ ID NOs: 29 and 30, or SEQ ID NOs: 32 and 33. It is understood that these primer pairs are examples of suitable primers for use in the context of the invention. In other examples, each primer can be between 10 and 40 nucleotides and together the pair of primers can flank a region of at least 10, 20, 30, 40, 50, 60, 70, 80, 90, 100, 120, 130, 140, 150, 200, 250, 300 bp in length that includes one or more CpG dinucleotides in a CpG island of interest. Primer pairs can be modified in various ways, such as by chemical modification of a base, and still be useful in the context of the invention. The only requirement is that such primers function to assay for methylation of a given CpG island. Thus, for example, alternate primers can be selected or the provided primers can be modified or provided in degenerate form to account for target sequence polymorphisms within a given population, so long as the primers are still suitable for assaying modification of CpG islands associated with the genes disclosed herein.

Like the target sequences, the primer pairs can be isolated or purified in accordance with methods known in the art. Alternatively, they can be synthesized using routine methods.

The primers can be part of a kit. Preferably, the kit comprises at least three pairs of primers, wherein each primer pair is specific for a CpG island associated with a different gene. However, the kit can comprise additional primer pairs, such as primer pairs for other CpG islands associated with the same gene or primer pairs for amplifying CpG islands associated with 4, 5, 6, 7, 8, 9, 10, 11, 12, 13, 14, 15, 16, 17, 18, 19, 20, 21, 22, 23, 24, 25, 26, 27 or even more genes. The kit can further comprise one or more reagents for assaying for the methylation of CpG islands, instructions for use, and/or other components as are typically found in kits. For example, the kit can comprise a buffer suitable for (a) isolating genomic DNA comprising a target sequence from a biological sample, (b) amplifying a portion of the target sequence, and/or (c) deaminating a target sequence. In embodiments directed to the evaluation of prostate cancer, a kit can comprise one or more buffers suitable for preparing genomic DNA from serum and/or urine samples.

### EXAMPLES

The following examples serve to illustrate the invention. The examples are not intended to limit the scope of the invention.

### Example 1

This example demonstrates the determination of the methylation status of markers based on methylation-specific PCR amplification. Paraffin-embedded prostate tissues were obtained following radical prostatectomies. The tissue samples were sectioned into 23 10-micron sections and slide 1, 12, and 23 were stained using hematoxylin and eosin (H&E). Using the H&E slides as guide, the areas corresponding to the tumor tissues were microdissected from the unstained slides. The remaining tissues were recovered to use as a normal paired sample. Following deparaffinization using two xylene extractions and two ethanol washes, the DNA was isolated from the tumor tissue and surrounding normal tissues using standard proteinase K digest for 5 days at 50° C., followed by ethanol precipitation (Current Protocols in Molecular Biology, edited by Ausubel, et al., Wiley-Interscience (New York 1988, revised 1988-2006)). The DNA was resuspended in TE8 and the quality and quantity of the DNA was assessed by agarose gel electrophoresis using concentration and size standards as reference. Following denaturation in the presence of 0.3 M NaOH, the DNA was treated with 2.5 M sodium metabisulfite, pH 5.5, in the presence of 1 mM hydroquinone at a concentration of 1 μg of DNA/500 μl. The reaction was incubated in a thermocycler for a total of 8 cycles (95° C. for 5 minutes; 55° C. for 115 minutes).

Following bisulfite treatment, the DNA was purified using the QIAEX II® purification kit (Qiagen, Valencia, Calif.) according to the manufacturer's recommendations and eluted in 50 μl of TE8. Sodium hydroxide (5.5 μl of 2 N) was added, and the DNA was incubated at RT for 15 min. The DNA was then precipitated with 3 volumes of ethanol and 0.3 volumes of 5 M NH₄OAC. The DNA was resuspended in 50 μl of TE8 and stored at −20° C.

In order to determine if a specific CpG position is methylated in genomic DNA isolated from tumor tissue, methylation-specific polymerase chain reaction (PCR) was performed, using primers designed to overlap the position of the CpG island of interest. All PCR reactions were performed in a MASTERCYCLER® (Eppendorf, Westbury, N.Y.) for 42 cycles of 95° C. for 20 seconds, 60° C. for 20 seconds, and 72° C. for 20 seconds. Each reaction was carried out in 30 μl of 1× TAKARA HOT START TAQ PCR buffer containing 1.5 mM magnesium chloride, 0.25 mM dNTPs, 25 picomoles of each primer, and 0.5 units of TAKARA HOT START TAQ enzyme (Clontech, Mountain View, Calif.). The primers used for each CpG island and the size of the product are shown in Table 1, wherein “F” indicates forward primer, “R” indicates reverse primer, and “m” indicates primer specific for methylated templates.

TABLE 1

| Gene | | Annealing | Product |
|---|---|---|---|
| associated with | | temperature | size |
| CpG island | Primer sequences | (° C.) | (bp) |
| ARHGEF10 | mF: ACGCGTGCGTAGTTTGGGTC | 60 | 112 |
| | [SEQ ID NO: 16] | | |
| | mR: | | |
| | CTACCGATTTCTCGACAACGCC | | |
| | [SEQ ID NO: 17] | | |
| MOXD1 | mF: | 60 | 120 |
| | GAGGAGTAGAACGAGGAGCGGT | | |
| | [SEQ ID NO: 18] | | |
| | mR: | | |
| | TACAAACGACCCGAAACGAAA | | |
| | [SEQ ID NO: 19] | | |
| NT5E | mF: | 60 | 119 |
| | TGGTTTATCGTGTATAAGGGCGT | | |
| | [SEQ ID NO: 20] | | |
| | mR: | | |
| | CGACTCCTCTCTCAATCCGAAA | | |
| | [SEQ ID NO: 21] | | |
| PHIP | mF: | 60 | 112 |
| | TAGCGTTTCGTACGCGGTATAG | | |
| | [SEQ ID NO: 22] | | |
| | mR: | | |
| | CAACCGACGACTTAAACTCCGA | | |
| | [SEQ ID NO: 23] | | |
| PTPRK | mF: TCGTAGTTTGGGTTTCGTCGTT | 60 | 120 |
| | [SEQ ID NO: 24] | | |
| | mR: | | |
| | GACTTACCGAACTCTCGCCTCT | | |
| | [SEQ ID NO: 25] | | |

The products of the PCR reactions were separated on 8% acrylamide gel. Only templates that exhibited methylation at all or most of the CpG dinucleotides that were present within the primers could serve as efficient templates for the amplification reactions. Control reactions were performed using fully methylated templates that were methylated in vitro using SSS1 (CpG) methylase (New England Biolabs, Ipswich, Mass.) according to the manufacturer's protocol. All primer pairs listed in Table 1 yielded a product of the correct size from fully methylated control template. Two negative controls (water and DNA isolated from white blood cells) are included for each target PCR amplification, which do not yield a PCR product. When a CpG island is methylated in a DNA sample, an amplification product of the expected size is obtained. This example demonstrates that the above primers can be used to assay for methylation of CpG islands in prostate cancer and that the CpG islands exhibit methylation in prostate cancer.

### Example 2

This example demonstrates the determination of the methylation pattern of multiple CpG islands associated with NT5E, PTPRK and MOXD1 using terminator-coupled linear amplification. From tumor DNA prepared as described in Example 1, fragments of the CpG islands associated with NT5E, PTPRK and MOXD1 are amplified individually using the nF1 and nR1 primers shown below in Table 2 for each indicated CpG island. The nF1 and nR1 primers amplify the indicated target sequences regardless of the templates' methylation status because the primer annealing sites do not contain any CpG dinucleotides. The amplification reactions are performed for 42 cycles as described in example 1 except for the annealing temperature, which is lowered to 58° C. An aliquot of the amplification reaction is separated on an 8% acrylamide gel to verify that fragments of the appropriate length are obtained (359 bp for MOXD1, 286 bp for PTPRK, 319 bp for NT5E). The products of the PCR reaction are treated with EXOSAP-IT® enzyme according to the recommendations of the manufacturer (Affymetrix, Santa Clara Calif.). The amplification products are used directly or purified further using AMICON® 100K 0.5 mL filters (Millipore, Billerica, Mass.) and/or ethanol precipitation.

TABLE 2

| Gene | | | Size of |
|---|---|---|---|
| associated | | Annealing | mF1/mR1 |
| with CpG | | temperature | product |
| island | Primer sequences | (° C.) | (bp) |
| MOXD1 | nF1: GTTAGGTGAGAGAGAGTTGTGG | 58 | 359 |
| | [SEQ ID NO: 26] | | |
| | nR1: CTACTCCTACTATAAAAACTACTCC | | |
| | [SEQ ID NO: 27] | | |
| | mR2F: 6FAM-AACTCGAACCGAACCTATCCG | | |
| | [SEQ ID NO: 28] | | |
| NT5E | nF1: GATTAGTATTAGGGTATTATTTGGT | 58 | 319 |
| | [SEQ ID NO: 29] | | |
| | nR1: ACTCCTCTAAATACTCCCATCTC | | |
| | [SEQ ID NO: 30] | | |
| | mF2F: 6FAM-ATCGTGTATAAGGGCGTCGAG | | |
| | [SEQ ID NO: 31] | | |
| PTPRK | nF1: GGAGGTTGTTTTTGTTAGTTAAGAG | 58 | 286 |
| | [SEQ ID NO: 32] | | |
| | nR1: CCTCTCAAATTCTAAATTCTAAAACA | | |
| | [SEQ ID NO: 33] | | |
| | mR1F: 6FAM-TCTAAAACAAAACAACGAA | | |
| | CTACG [SEQ ID NO: 34] | | |

Each amplification product (10 nanograms) is subjected to terminator-coupled linear amplification (TCLA) using THERMOSEQUENASE cycle sequencing kit (Affymetrix) and the corresponding nested primers shown in Table 2 (mR2F, mF2F, and mR1F primers for MOXD1, NT5E, PTPRK respectively), which are tagged at the 5′ terminus with carboxyfluoroscein (FAM).

For the amplified NT5E CpG island fragment, the TCLA reaction is performed in 1× ThermoSequenase™ buffer, 60 μM dATP, 15 μM dCTP, 60 μM dGTP, 60 μM dTTP, 1 μM ddCTP, 1.5 picomoles of NT5E-mF2F primer (see Table 2), and 2 units of the ThermoSequenase™ DNA polymerase. For the amplified MOXD1 CpG island fragment and the PTPRK CpG island fragment, the TCLA reactions are performed in 1× THERMOSEQUENASE buffer, 60 μM dATP, 60 μM dCTP, 15 μM dGTP, 60 μM dTTP, 1 μM ddGTP, 1.5 picomoles of MOXD1 mR2F primer (Table 2) or PTPRK mR1F primer (Table 2), and 2 units of the ThermoSequenase™ DNA polymerase. Reactions are performed in a MASTERCYCLER® thermocycler (Eppendorf) for 30 cycles of 95° C. for 15 seconds, and 60° C. for 60 seconds. Following amplification, the TCLA reaction products are subjected to ethanol precipitation and resuspended in 30 μl of deionized formamide. One microliter of GENESCAN® 500 LIZ standard (Applied Biosystems, Foster City, Calif.) is added to each tube and the DNA is separated using the ABI PRISM 310 Genetic Analyzer (Applied Biosystems) using the GENESCAN® application. The data is analyzed using the GENEMAPPER® software (Applied Biosystems) to provide information about TCLA reaction product length.

The nF1 and nR1 primers shown in Table 2 are used to amplify target sequences of CpG islands associated with NT5E, PTPRK and MOXD1 from tumor DNA. Subsequently, the amplified target sequences and FAM-tagged primers are used in TCLA reactions to generate FAM-tagged products having different lengths (bp). The lengths of the TCLA products indicate (i) the presence and/or the positions of methylated cytosines in the CpG islands associated with NT5E, PTPRK and MOXD1 and (ii) provide information about the efficiency of the deamination reaction, since an incomplete deamination results in TCLA product with lengths that differ than what is expected based on the positions of the CpG dinucleotides within the sequence.

### Example 3

This example demonstrates the determination of the methylation status of CpG islands associated with NT5E, PTPRK and MOXD1 by quantitative PCR using TAQMAN® assays. From tumor DNA prepared as described in Example 1, fragments of the CpG islands associated with each of NT5E, PTPRK and MOXD1 are amplified individually using the qF1 and qR1 primers and the TAQMAN® (PM1) Probes shown below in Table 3. The TAQMAN® (PM1) probes are tagged at the 5′ terminus with FAM and at the 3′ terminus with BHQ1 quencher (Biosearch Technologies, Novato, Calif.).

The amplification reactions are performed for 50 cycles in a REALPLEX⁴ Mastercycler (Eppendorf, Westbury, N.Y.) for 50 cycles of 95° C. for 15 seconds, 69° C. for 30 seconds, and 64° C. for 20 seconds. Each reaction is carried out in 25 μl of 1× TAKARA TAQ PCR buffer supplemented with magnesium chloride (final concentration of MgCl₂ is 2.5 mM), 0.25 mM dNTPS, 12.5 picomoles of each primer, 5 picomoles of the probe, and 0.5 units of TAKARA HOT START TAQ enzyme (Clontech, Mountain View, Calif.). For negative and positive controls, bisulfite conversion is performed on 1 μg of DNA isolated from a lymphoblastoid cell line with or without treatment with SSSI methylase. Amplification reactions corresponding to 1, 5, 10, 20 ng of DNA (pre-bisulfite conversion concentration) are performed in triplicates. The fractional cycle number at which FAM fluorescence passes the threshold (CT FAM) is determined for each DNA sample. The threshold is a level set just above baseline yet sufficiently low to be in the exponential growth region of amplification. Tumors with CT FAM higher than the CT FAM observed for the unmethylated blood DNA for each marker are considered methylated at the corresponding locus.

TABLE 3

| Gene | | | Size of |
|---|---|---|---|
| associated | | Annealing | qF1/qR1 |
| with | | temperature | product |
| CpG island | Primer sequences | (° C.) | (bp) |
| MOXD1 | qF1: GGGTCGTTCGTGGGTTATTTC | 69 | 105 |
| | [SEQ ID NO: 35] | | |
| | qR1: CGCCTCCAAATACGCACTAC | | |
| | [SEQ ID NO: 36] | | |
| | PM1: FAM-TACGACGATGTCGGCGGAC | | |
| | GTTA-BHQ1 [SEQ ID NO: 37] | | |
| NT5E | qF1: GCTTTGCGCTACGATGCTATG | 69 | 101 |
| | [SEQ ID NO: 38] | | |
| | qR1: ACTCTGCCATCCGCTGCTTT | | |
| | [SEQ ID NO: 39] | | |
| | PM1: FAM-TAAGATTCGAGCTCGCGC | | |
| | TCGG-BHQ1 [SEQ ID NO: 40] | | |
| PTPRK | qF1: TCGTAGTTTGGGTTTCGTCGTT | 69 | 120 |
| | [SEQ ID NO: 41] | | |
| | qR1: GACTTACCGAACTCTCGCCTCTC | | |
| | [SEQ ID NO: 42] | | |
| | PM1: FAM-TTCGGAGGGAGCGAGCGAG AAAG- | | |
| | BHQ1 [SEQ ID NO: 43] | | |

An aliquot of the amplification reaction is separated on an 8% acrylamide gel to verify that fragments of the appropriate length are obtained (105 bp for MOXD1, 120 bp for PTPRK, 101 bp for NT5E).

All references, including publications, patent applications, and patents, cited herein are hereby incorporated by reference to the same extent as if each reference were individually and specifically indicated to be incorporated by reference and were set forth in its entirety herein.

The use of the terms “a,” “an,” “the,” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

1. A method of determining the methylation status of one or more CpG islands indicative of prostate cancer in a human male, which method comprises
isolating or amplifying genomic DNA from a biological sample from a human male undergoing prostate cancer evaluation; and
assaying the genomic DNA for the methylation status of one or more CpG islands associated with at least one gene selected from the group consisting of ARHGEF10 (Rho guanine nucleotide exchange factor 10 gene), NT5E (5′-nucleotidase, ecto (CD73) gene), MOXD1 (monooxygenase, DBH-like 1 gene), PTPRK (protein tyrosine phosphatase, receptor type K gene), and PHIP (pleckstrin homology domain interacting protein gene),
wherein the genomic DNA includes genomic DNA, fragments of genomic DNA, or a combination thereof, and wherein a positive methylation status of the one or more assayed CpG islands is indicative of prostate cancer in the human male.

2. The method of claim 1, wherein the method comprises assaying the isolated or amplified genomic DNA for methylation of CpG islands associated with at least two genes selected from the group consisting of ARHGEF10, NT5E, MOXD1, PTPRK, and PHIP and wherein a positive methylation status of the assayed CpG islands is indicative of prostate cancer.

3. The method of claim 1, wherein the method comprises assaying the isolated or amplified genomic DNA for methylation of CpG islands associated with at least three genes selected from the group consisting of ARHGEF10, NT5E, MOXD1, PTPRK, and PHIP and wherein a positive methylation status of the assayed CpG islands is indicative of prostate cancer.

4. The method of claim 1, wherein the method comprises assaying the isolated or amplified genomic DNA for methylation of one or more CpG islands associated with at least one gene selected from the group consisting of NT5E, MOXD1, and PTPRK and wherein a positive methylation status of the one or more assayed CpG islands is indicative of prostate cancer.

5. The method of claim 1, wherein the method comprises assaying the isolated or amplified genomic DNA for methylation of CpG islands associated with NT5E, MOXD1, and PTPRK and wherein positive methylation status of the CpG islands is indicative of prostate cancer.

6. The method of claim 1, wherein the method further comprises assaying the isolated or amplified genomic DNA for methylation of one or more CpG islands associated with at least one gene that is known to be methylated in prostate cancer and that is known not to be detectably methylated or methylated at a lower level in benign prostate hyperplasia (BPH), and wherein positive methylation status of the assayed one or more CpG islands is indicative of prostate cancer.

7. The method of claim 6, wherein the one or more CpG islands associated with at least one gene that is known to be methylated in prostate cancer and that is known not to be detectably methylated or methylated at a lower level in BPH includes a CpG islands associated with a gene selected from the group consisting of glutathione S-transferase P1 (GSTP1), adenomatosis polyposis coli (APC), Cub and Sushi multiple domains1 (CSMD1), tumor necrosis factor receptor superfamily member 10A (TNFRSF10A) tumor necrosis factor receptor superfamily member 10B (TNFRSF10B), tumor necrosis factor receptor superfamily member 10C (TNFRSF10C), tumor necrosis factor receptor superfamily 10D (TNFRSF10D), secreted frizzled-related protein 1 (SFRP1), secreted frizzled-related protein 2 (SFRP2), dickkopf homolog 3 (DKK3), prostaglandin-endoperoxide synthase 2 (PTGS2), cyclin-dependent kinase inhibitor 1C (CDKN1C/p57), Ras association (RalGDS/AF-6) domain family 1 (RASSF1), and G-protein coupled receptor 62 (GPR62).

8. The method of claim 6, wherein the one or more CpG islands associated with at least one gene that is known to be methylated in prostate cancer and that is known not to be detectably methylated or methylated at a lower level in BPH includes a CpG island associated with GSTP1.

9. The method of claim 1, wherein the assaying for methylation of a CpG island comprises amplifying a target sequence that includes at least one CpG dinucleotide in a target sequence selected from the group consisting of SEQ ID NOs: 2 or 3 [ARHGEF10], SEQ ID NOs: 5 or 6 [MOXD1], SEQ ID NOs: 8 or 9 [NT5E], SEQ ID NOs: 11 or 12 [PHIP], and SEQ ID NOs: 14 or 15 [PTPRK].

10. The method of any of claims 1, wherein the biological sample is a tissue sample or biological fluid.

11. The method of claim 1, wherein the biological sample is whole blood, blood plasma, blood serum, saliva, cells, or needle aspirate.

12. The method of claim 1, wherein the biological sample is urine.

13. The method of claim 1, wherein the biological sample is prostate tissue.

14. A method of treating prostate cancer in a cancer in a human male, wherein the method comprises determining the methylation status of one or more CpG islands indicative of prostate cancer in a human male according to claim 1, wherein the one or more assayed CpG islands are methylated and wherein the method further comprises treating the human male for prostate cancer.

15. A pair of isolated, purified, or synthesized nucleic acid molecules, wherein each nucleic acid molecule consists essentially of SEQ ID NOs: 2 and 3 [ARHGEF10], respectively, SEQ ID NOs: 5 and 6 [MOXD1], respectively, SEQ ID NOs: 8 and 9 [NT5E], respectively, SEQ ID NOs: 11 and 12 [PHIP], respectively, SEQ ID NOs: 14 and 15 [PTPRK], respectively, SEQ ID NOs: 16 and 17 [ARHGEF10], respectively, SEQ ID NOs: 18 and 19 [MOXD1], respectively, SEQ ID NOs: 26 and 27 [MOXD1], respectively, SEQ ID NOs: 20 and 21 [NT5E], respectively, SEQ ID NOs: 29 and 30 [NT5E], respectively, SEQ ID NOs: 22 and 23 [PHIP], respectively, SEQ ID NOs: 24 and 25 [PTPRK], respectively, or SEQ ID NOs: 32 and 33 [PTPRK], respectively.

16. A method of assessing the efficacy of treatment of prostate cancer by assaying for reduced methylation of CpG islands in a human male, which method comprises
isolating or amplifying genomic DNA from a first biological sample and a second biological from a human male, wherein the first biological sample is from the male before or during a treatment and the second biological is from the male after the treatment;
assaying the genomic DNA from the first biological for methylation of one or more CpG islands associated with at least one gene selected from the group consisting of ARHGEF10 (Rho guanine nucleotide exchange factor 10 gene), NT5E (5′-nucleotidase, ecto (CD73) gene), MOXD1 (monooxygenase, DBH-like 1 gene), PTPRK (protein tyrosine phosphatase, receptor type K gene), and PHIP (pleckstrin homology domain interacting protein gene);
assaying the genomic DNA from the second biological for methylation of one or more CpG islands associated with at least one gene selected from the group consisting of ARHGEF10, NT5E, MOXD1, PTPRK, and PHIP; and
determining whether there is a change in the methylation status of the one or more CpG islands in the second sample relative to the first sample;
wherein the genomic DNA includes genomic DNA, fragments of genomic DNA, or a combination thereof, and wherein (i) maintenance or an increase of methylation of the one more CpG islands in the second sample relative to the first sample indicates that the treatment is ineffective or having reduced efficacy and (ii) an absence or decrease of methylation of the one more CpG islands in the second sample relative to the first sample indicates that the treatment is effective.

17. The method of claim 16, wherein the method comprises assaying the isolated or amplified genomic DNA of the first and second biological samples for methylation of CpG islands associated with at least two genes selected from the group consisting of ARHGEF10, NT5E, MOXD1, PTPRK, and PHIP.

18. The method of claim 16, wherein the method comprises assaying the isolated or amplified genomic DNA of the first and second biological samples for methylation of CpG islands associated with at least three genes selected from the group consisting of ARHGEF10, NT5E, MOXD1, PTPRK, and PHIP.

19. The method of claim 18, wherein the method comprises assaying the isolated or amplified genomic DNA of the first and second biological samples for methylation of CpG islands associated with at least one gene selected from the group consisting of NT5E, MOXD1, and PTPRK.


SEQUENCE LISTING (43 sequences)

SEQ ID NO: 1 - DNA, 1300 bp, Homo sapiens
aaaaactgcctcggaaacgtggctgaagtcatcgcatttccaaacgcccaagccttggctggtccagggcagctggcgcggaagacccggaacgccctgtgggcagggccagctgcggacggagggacgggggctcaagggcgggtcgggtggggtcccgacgccccctcctcggctgggcgcaccccgggtgtccgcagggaggggcgatgttctgggggggggagcgtctgcccgacgccccctccctcaggtgagctcagcccgtgggggtccgcagaaagggcggtgctctgggagagggttagggtctgcccggcgcaccctccccaaggggcgcagctctgagccgggagcaggctctgcggggaggatccagatgttgggggcggggagggcactcggtctgcccagcgacccctcccgaagtgagcgcggcccggggtccgcggggcaggagatgatccgggggcgggagcctctgccgcgcgccccctcccacgcgtgcgcagcctgggccgggcgttgggcagcgggagggggcgcgggcggccacgtggcctcgaaggaatgcgcgggcgcgagggcagggcgctgccgagaaaccggcaggctctgcggccaatgggcgcccggaacggtctgggcccgccaatgacagcatggtgggtggagcaggccgtccgggcgggaggggctgggcgcatccctgtagccggcgggcgcgcgatccgggacggacggggtcgcgggggacgcgggggacgcgggggacggcggggaacggcgggggacggcggggaacagcgggggacggcggggaacagcgggggacggcggggaacagcaggggactgcgggtcgcgtcctggccgccgaggtgagtgcgggccccgcggaccgtgggtcccctcgcgggcttctggtttcctttgagagttgcacttctgcccggctcccccgaaatcgctgggtcccacccggaggtggctgcgcccccgaccccctgcgcagatgtttcagggaaggggctgagaccccctccccccgagcagctcccccttccgcggcgcccccggcccctcaggataacggcgcgggcctgggcggggggagcagggactggcagcagggacccctggccccaggggcaggagcgaactccgatcggcggaaggagcccgaggggcgagctggcttcgcctggggggtcgtgcagtaacttagattttccggtaaaggagaagaatgcggtccgcctcaggatgcagagaaggaccggtgtaaggaaagccggcgt

SEQ ID NO: 2 - DNA, 1300 bp, Artificial Sequence
  Synthetic Sequence
aaaaattgtttcggaaacgtggttgaagttatcgtatttttaaacgtttaagttttggttggtttagggtagttggcgcggaagattcggaacgttttgtgggtagggttagttgcggacggagggacgggggtttaagggcgggtcgggtggggtttcgacgtttttttttcggttgggcgtatttcgggtgttcgtagggaggggcgatgttttgggggggggagcgtttgttcgacgtttttttttttaggtgagtttagttcgtgggggttcgtagaaagggcggtgttttgggagagggttagggtttgttcggcgtattttttttaaggggcgtagttttgagtcgggagtaggttttgcggggaggatttagatgttgggggcggggagggtattcggtttgtttagcgatttttttcgaagtgagcgcggttcggggttcgcggggtaggagatgattcgggggcgggagtttttgtcgcgcgtttttttttacgcgtgcgtagtttgggtcgggcgttgggtagcgggagggggcgcgggcggttacgtggtttcgaaggaatgcgcgggcgcgagggtagggcgttgtcgagaaatcggtaggttttgcggttaatgggcgttcggaacggtttgggttcgttaatgatagtatggtgggtggagtaggtcgttcgggcgggaggggttgggcgtatttttgtagtcggcgggcgcgcgattcgggacggacggggtcgcgggggacgcgggggacgcgggggacggcggggaacggcgggggacggcggggaatagcgggggacggcggggaatagcgggggacggcggggaatagtaggggattgcgggtcgcgttttggtcgtcgaggtgagtgcgggtttcgcggatcgtgggttttttcgcgggtttttggttttttttgagagttgtatttttgttcggttttttcgaaatcgttgggttttattcggaggtggttgcgttttcgattttttgcgtagatgttttagggaaggggttgagatttttttttttcgagtagttttttttttcgcggcgttttcggttttttaggataacggcgcgggtttgggcggggggagtagggattggtagtagggatttttggttttaggggtaggagcgaatttcgatcggcggaaggagttcgaggggcgagttggtttcgtttggggggtcgtgtagtaatttagatttttcggtaaaggagaagaatgcggttcgttttaggatgtagagaaggatcggtgtaaggaaagtcggcgt

SEQ ID NO: 3 - DNA, 1300 bp, Artificial Sequence
  Synthetic Sequence
aaaaattgttttggaaatgtggttgaagttattgtatttttaaatgtttaagttttggttggtttagggtagttggtgtggaagatttggaatgttttgtgggtagggttagttgtggatggagggatgggggtttaagggtgggttgggtggggttttgatgttttttttttggttgggtgtattttgggtgtttgtagggaggggtgatgttttgggggggggagtgtttgtttgatgtttttttttttaggtgagtttagtttgtgggggtttgtagaaagggtggtgttttgggagagggttagggtttgtttggtgtattttttttaaggggtgtagttttgagttgggagtaggttttgtggggaggatttagatgttgggggtggggagggtatttggtttgtttagtgattttttttgaagtgagtgtggtttggggtttgtggggtaggagatgatttgggggtgggagtttttgttgtgtgtttttttttatgtgtgtgtagtttgggttgggtgttgggtagtgggagggggtgtgggtggttatgtggttttgaaggaatgtgtgggtgtgagggtagggtgttgttgagaaattggtaggttttgtggttaatgggtgtttggaatggtttgggtttgttaatgatagtatggtgggtggagtaggttgtttgggtgggaggggttgggtgtatttttgtagttggtgggtgtgtgatttgggatggatggggttgtgggggatgtgggggatgtgggggatggtggggaatggtgggggatggtggggaatagtgggggatggtggggaatagtgggggatggtggggaatagtaggggattgtgggttgtgttttggttgttgaggtgagtgtgggttttgtggattgtgggtttttttgtgggtttttggttttttttgagagttgtatttttgtttggtttttttgaaattgttgggttttatttggaggtggttgtgtttttgattttttgtgtagatgttttagggaaggggttgagattttttttttttgagtagtttttttttttgtggtgtttttggttttttaggataatggtgtgggtttgggtggggggagtagggattggtagtagggatttttggttttaggggtaggagtgaattttgattggtggaaggagtttgaggggtgagttggttttgtttggggggttgtgtagtaatttagattttttggtaaaggagaagaatgtggtttgttttaggatgtagagaaggattggtgtaaggaaagttggtgt

SEQ ID NO: 4 - DNA, 1050 bp, Homo sapiens
cttctcccttcccatcagggatgaagcggggtgtattgcaggtgtgtaggcgggggggcgtctattaagtggggcgctggggacaggaaagtgttcaaagaaggaaacctgcctggggtcatccgtgctcggcttccctaccgcgcgccttctggggctccgggagagcaggcgctgcccgcggaggcgagagtgagcgtggccgggggcgcggggctgcgccaggtgagagagagctgcgggccccggggacgacccgcacctgcgccctctctggggtccggacgggaccgggctcggccgggcgggctccgggaggagacgcgcttacctggaggtagggccgcccgtgggccaccccgcccacgacgatgtcggcggacgccatggccccggtgggcgagaagccgaagcccacgtagcctgcagtgcgcacctggaggcggaaggcgatctggctgccccgctggctccagcccagccagtacttgccctccgagtccaggagggtccggtgcggataggttcggcccgagccccccgccgccgtcccggggagcagcccccacagcaggagcagcggccagcagcacatcctcgggcgcctcctgcccgccggtaccggcctccagccgctggggagtgaggagcagaacgaggagcggcagcggcggcgagcgggagcccagggaccgcccctcgggccggccgcgcgcccgcccgggaggggcaggacccgctctccccgccccgggccgcctgcagggcgcgccgcgtcccgtccggtcccgtcccgttccgtcccgtcgggtcccagccccgccaactctcaccctgcccggcccttccagcggatccacttgcagcccccgacccctgacttggtccccgaggtcggggctcggtgggaaggaaagcgaggggcttcccggaaagaaagggaggaggaggcaggaggaggcagacgccgagagaggagaaaaggaggccagaggcagagggaaaaggaggagggcaagagaatcctagagcgggaagagcgtgggctct

SEQ ID NO: 5 - DNA, 1050 bp, Artificial Sequence
  Synthetic Sequence
tttttttttttttattagggatgaagcggggtgtattgtaggtgtgtaggcgggggggcgtttattaagtggggcgttggggataggaaagtgtttaaagaaggaaatttgtttggggttattcgtgttcggtttttttatcgcgcgttttttggggtttcgggagagtaggcgttgttcgcggaggcgagagtgagcgtggtcgggggcgcggggttgcgttaggtgagagagagttgcgggtttcggggacgattcgtatttgcgttttttttggggttcggacgggatcgggttcggtcgggcgggtttcgggaggagacgcgtttatttggaggtagggtcgttcgtgggttatttcgtttacgacgatgtcggcggacgttatggtttcggtgggcgagaagtcgaagtttacgtagtttgtagtgcgtatttggaggcggaaggcgatttggttgtttcgttggttttagtttagttagtatttgtttttcgagtttaggagggttcggtgcggataggttcggttcgagtttttcgtcgtcgtttcggggagtagtttttatagtaggagtagcggttagtagtatattttcgggcgttttttgttcgtcggtatcggtttttagtcgttggggagtgaggagtagaacgaggagcggtagcggcggcgagcgggagtttagggatcgtttttcgggtcggtcgcgcgttcgttcgggaggggtaggattcgtttttttcgtttcgggtcgtttgtagggcgcgtcgcgtttcgttcggtttcgtttcgtttcgtttcgtcgggttttagtttcgttaatttttattttgttcggtttttttagcggatttatttgtagttttcgatttttgatttggttttcgaggtcggggttcggtgggaaggaaagcgaggggtttttcggaaagaaagggaggaggaggtaggaggaggtagacgtcgagagaggagaaaaggaggttagaggtagagggaaaaggaggagggtaagagaattttagagcgggaagagcgtgggtttt

SEQ ID NO: 6 - DNA, 1050 bp, Artificial Sequence
  Synthetic Sequence
tttttttttttttattagggatgaagtggggtgtattgtaggtgtgtaggtgggggggtgtttattaagtggggtgttggggataggaaagtgtttaaagaaggaaatttgtttggggttatttgtgtttggtttttttattgtgtgttttttggggttttgggagagtaggtgttgtttgtggaggtgagagtgagtgtggttgggggtgtggggttgtgttaggtgagagagagttgtgggttttggggatgatttgtatttgtgttttttttggggtttggatgggattgggtttggttgggtgggttttgggaggagatgtgtttatttggaggtagggttgtttgtgggttattttgtttatgatgatgttggtggatgttatggttttggtgggtgagaagttgaagtttatgtagtttgtagtgtgtatttggaggtggaaggtgatttggttgttttgttggttttagtttagttagtatttgttttttgagtttaggagggtttggtgtggataggtttggtttgagttttttgttgttgttttggggagtagtttttatagtaggagtagtggttagtagtatatttttgggtgttttttgtttgttggtattggtttttagttgttggggagtgaggagtagaatgaggagtggtagtggtggtgagtgggagtttagggattgttttttgggttggttgtgtgtttgtttgggaggggtaggatttgttttttttgttttgggttgtttgtagggtgtgttgtgttttgtttggttttgttttgttttgttttgttgggttttagttttgttaatttttattttgtttggtttttttagtggatttatttgtagtttttgatttttgatttggtttttgaggttggggtttggtgggaaggaaagtgaggggttttttggaaagaaagggaggaggaggtaggaggaggtagatgttgagagaggagaaaaggaggttagaggtagagggaaaaggaggagggtaagagaattttagagtgggaagagtgtgggtttt

SEQ ID NO: 7 - DNA, 1300 bp, Homo sapiens
agcgtcacatctcagtgagcttcggctctcacagcccgaaactgcaaggagctcctctgcccggcctctctttactcctcctctctgcccctcagctcgctcatctttcttcccgccccctctcttttccttctttggttctttgaagtgatgagctagcgcaaccacaaaccatacattccttttgtagaaaaacccgtgcctcgaatgaggcgagactcagagaggacccaggcgcggggcggacccctccaattccttcctcgcgcccccgaaagagcggcgcaccagcagccgaactgccggcgcccaggctccctggtccggccgggatgcggccggtacccgctccccgccgggaacaacctctccactcttcctgcagggagctggtgccagccgacagccgcgccagggccgctccgggtaccagggtcggatcgggtgacgtcgcgaacttgcgcctggccgccaagccggcctccaggctgaagaaggacccgccccggccttgacccgggccccgcccctccagccggggcaccgagccccggccctagctgctcgcccctactcgccggcactcgcccggctcgcccgctttcgcacccagttcacgcgccacagctatgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggtaagacccgagcccgcgcccgggatagtagtcccggactgagagaggagccgggctggaaaagcagcggatggcagagtgtggcaagcctaggtccagggcgcggagagatgtggggataaagtgagactccggccagtgtgccagctggatgcatagaagtcccttggacgattcgtcttaaacggacgttattgcgcccccactatgagatgggagtatttagaggagcaggacccagtcctgcctgcgaggggcttaaggtggggtgcgtagaagcactgacagttatccagagctagggactgaatcggatccctcacgcagctctc

SEQ ID NO: 8 - DNA, 1300 bp, Artificial Sequence
  Synthetic Sequence
agcgttatattttagtgagtttcggtttttatagttcgaaattgtaaggagtttttttgttcggtttttttttattttttttttttgttttttagttcgtttattttttttttcgtttttttttttttttttttttggttttttgaagtgatgagttagcgtaattataaattatatattttttttgtagaaaaattcgtgtttcgaatgaggcgagatttagagaggatttaggcgcggggcggatttttttaatttttttttcgcgttttcgaaagagcggcgtattagtagtcgaattgtcggcgtttaggttttttggttcggtcgggatgcggtcggtattcgtttttcgtcgggaataatttttttattttttttgtagggagttggtgttagtcgatagtcgcgttagggtcgtttcgggtattagggtcggatcgggtgacgtcgcgaatttgcgtttggtcgttaagtcggtttttaggttgaagaaggattcgtttcggttttgattcgggtttcgtttttttagtcggggtatcgagtttcggttttagttgttcgtttttattcgtcggtattcgttcggttcgttcgttttcgtatttagtttacgcgttatagttatgtgttttcgagtcgcgcgggcgttcgcgacgttatttttcgttttgggcgcggtgttgtggtttgcggttggcgtttgggagtttacgattttgtatattaacgacgtgtatagtcggttggagtagattagcgaggattttagtaagtgcgttaacgttagtcgttgtatgggtggcgtggttcggttttttattaaggtttagtagattcgtcgcgtcgaatttaacgtgttgttgttggacgtcggcgattagtattagggtattatttggtttatcgtgtataagggcgtcgaggtggcgtattttatgaacgttttgcgttacgatgttatggtaagattcgagttcgcgttcgggatagtagtttcggattgagagaggagtcgggttggaaaagtagcggatggtagagtgtggtaagtttaggtttagggcgcggagagatgtggggataaagtgagatttcggttagtgtgttagttggatgtatagaagttttttggacgattcgttttaaacggacgttattgcgtttttattatgagatgggagtatttagaggagtaggatttagttttgtttgcgaggggtttaaggtggggtgcgtagaagtattgatagttatttagagttagggattgaatcggattttttacgtagttttt

SEQ ID NO: 9 - DNA, 1300 bp, Artificial Sequence
  Synthetic Sequence
agtgttatattttagtgagttttggtttttatagtttgaaattgtaaggagtttttttgtttggtttttttttattttttttttttgttttttagtttgtttatttttttttttgtttttttttttttttttttttggttttttgaagtgatgagttagtgtaattataaattatatattttttttgtagaaaaatttgtgttttgaatgaggtgagatttagagaggatttaggtgtggggtggatttttttaattttttttttgtgtttttgaaagagtggtgtattagtagttgaattgttggtgtttaggttttttggtttggttgggatgtggttggtatttgttttttgttgggaataatttttttattttttttgtagggagttggtgttagttgatagttgtgttagggttgttttgggtattagggttggattgggtgatgttgtgaatttgtgtttggttgttaagttggtttttaggttgaagaaggatttgttttggttttgatttgggttttgtttttttagttggggtattgagttttggttttagttgtttgtttttatttgttggtatttgtttggtttgtttgtttttgtatttagtttatgtgttatagttatgtgtttttgagttgtgtgggtgtttgtgatgttattttttgttttgggtgtggtgttgtggtttgtggttggtgtttgggagtttatgattttgtatattaatgatgtgtatagttggttggagtagattagtgaggattttagtaagtgtgttaatgttagttgttgtatgggtggtgtggtttggttttttattaaggtttagtagatttgttgtgttgaatttaatgtgttgttgttggatgttggtgattagtattagggtattatttggtttattgtgtataagggtgttgaggtggtgtattttatgaatgttttgtgttatgatgttatggtaagatttgagtttgtgtttgggatagtagttttggattgagagaggagttgggttggaaaagtagtggatggtagagtgtggtaagtttaggtttagggtgtggagagatgtggggataaagtgagattttggttagtgtgttagttggatgtatagaagttttttggatgatttgttttaaatggatgttattgtgtttttattatgagatgggagtatttagaggagtaggatttagttttgtttgtgaggggtttaaggtggggtgtgtagaagtattgatagttatttagagttagggattgaattggattttttatgtagttttt

SEQ ID NO: 10 - DNA, 2050 bp, Homo sapiens
ctactaaacttctggttttagggaattacaggctttacacttctgcaaagatgtgttttagtaaatgcaagacgaagcactgaccaactatcatcacacatcagaatccttccaacaaaaaacccaggactgaatttaaggaaaacaaaataaacgacagagggggaaaaaataatgtcttgccacggtaccgcagcggctggatagcctgcttgtgaaatgctaatgccacttcggagcagttagtcaccagctattgtgtagggcaggagaaagccgagccggccgcgcgtgcagagcgagcaagcgaacgagcgagcgcgctctccctcttcgcgccgctcccgccgccgccgactctcgcgcgcccccgcgcccgcacggacgcgcgcgccggcccctcctcctccggccttgcactgcacaacactcatgacgtatctttatttctagcacattaacaaaatatcacaaataaattgtccgcagcccctgcggccccgaagtacgagtacccccggccactggcccccgcagaccccgcgccggcctcccaaccctccccatggcctttggagctttcacgttctagggccaagtttttgtctctgtaaaaaattgcgggaaattcaatttttattcgactcagggaaaagtttctttgctctgcgacgtgaatgtctcaccagattctggtaggtcctgggatgctccttcccggtccagtcggtgcgccggggcagcagctgcggggagaggacaccccgtgagcccggcccccggcccctacccgcccgctcccctcccccgcccgccccgcgccccggcggggaccccgagccccgcgccccgcgccctgccggcggcggcagcggcggcgcagcggcccagaggcggccgcgcggcggcgggacgcgccgggccgccgccgcccttacctccttctcggccacctcgcggatcagcacctgcaacaacaaagcggggagagctgagccccgcgccccgggccgcggccccgccgccgccgcctccctcccccacgcccgcgagcggcgagcgccggcccggcccgcgccgcgcgccgccgtacctgagccgcctgctgacagggtccatcttccaggaaccgggcgatgaggaagtagagctctgcgcgggagagagggacggggagacacacaggctgagcggtcgggcggcggggggcgggggaccgcgggcggaatcgcccggtgccagcggccccggcagccccccgacttacccgatcgcagctccgagaggcctttcctctcacaagacatgtttatgggtcacttcagggccgccgacgggacaccccgccgccgaggggaagcggggacggtgccgccgcctgccctatagctgtcagtgtgtgttcacgagccgagcttcggctccaccattcaagcaacggcggcggaggcggaggaggaggaggaggaaacaacaactctcaggcagcgactacggccgtggccgcctccgccgcggatccctccgccgcagaaaggagtccgccgccttcgcggcccagggctcggccccggctctggcccgcgcccccgccccccggcgctaaaaaaggagtgcctccgacccctcgtccccagcgctccgcacgcggcacagtgagacccccacccgctcctccccgcagggcgtgcgatttatttatttatttccagtcggagaagatgtcggagcccaagccgccggttggctggaaggcgctttctctgtggaggccgatagtggcagggagggggccggggacggttccgcggagggatctgacgcacacggagccgcagcacaggctctattcagcggcgctggctggagctgagatggaagttagtttctatgtagcagaaatatgaaacaaatgaagcaaaactgcccagagaggggaaatgccccaaggatgggtctcactcacgcgcgtacacagacacacacgcagagagcactctcacgctgggcaagctcgggatcgcgctacccttcccgagttgaatgatag

SEQ ID NO: 11 - DNA, 2050 bp, Artificial Sequence
  Synthetic Sequence
ttattaaatttttggttttagggaattataggttttatatttttgtaaagatgtgttttagtaaatgtaagacgaagtattgattaattattattatatattagaatttttttaataaaaaatttaggattgaatttaaggaaaataaaataaacgatagagggggaaaaaataatgttttgttacggtatcgtagcggttggatagtttgtttgtgaaatgttaatgttatttcggagtagttagttattagttattgtgtagggtaggagaaagtcgagtcggtcgcgcgtgtagagcgagtaagcgaacgagcgagcgcgtttttttttttcgcgtcgttttcgtcgtcgtcgattttcgcgcgttttcgcgttcgtacggacgcgcgcgtcggttttttttttttcggttttgtattgtataatatttatgacgtatttttatttttagtatattaataaaatattataaataaattgttcgtagtttttgcggtttcgaagtacgagtattttcggttattggttttcgtagatttcgcgtcggttttttaattttttttatggtttttggagtttttacgttttagggttaagtttttgtttttgtaaaaaattgcgggaaatttaatttttattcgatttagggaaaagtttttttgttttgcgacgtgaatgttttattagattttggtaggttttgggatgttttttttcggtttagtcggtgcgtcggggtagtagttgcggggagaggatatttcgtgagttcggttttcggtttttattcgttcgtttttttttttcgttcgtttcgcgtttcggcggggatttcgagtttcgcgtttcgcgttttgtcggcggcggtagcggcggcgtagcggtttagaggcggtcgcgcggcggcgggacgcgtcgggtcgtcgtcgtttttatttttttttcggttatttcgcggattagtatttgtaataataaagcggggagagttgagtttcgcgtttcgggtcgcggtttcgtcgtcgtcgttttttttttttacgttcgcgagcggcgagcgtcggttcggttcgcgtcgcgcgtcgtcgtatttgagtcgtttgttgatagggtttattttttaggaatcgggcgatgaggaagtagagttttgcgcgggagagagggacggggagatatataggttgagcggtcgggcggcggggggcgggggatcgcgggcggaatcgttcggtgttagcggtttcggtagtttttcgatttattcgatcgtagtttcgagaggtttttttttttataagatatgtttatgggttattttagggtcgtcgacgggatatttcgtcgtcgaggggaagcggggacggtgtcgtcgtttgttttatagttgttagtgtgtgtttacgagtcgagtttcggttttattatttaagtaacggcggcggaggcggaggaggaggaggaggaaataataatttttaggtagcgattacggtcgtggtcgttttcgtcgcggattttttcgtcgtagaaaggagttcgtcgttttcgcggtttagggttcggtttcggttttggttcgcgttttcgtttttcggcgttaaaaaaggagtgttttcgatttttcgtttttagcgtttcgtacgcggtatagtgagatttttattcgttttttttcgtagggcgtgcgatttatttatttatttttagtcggagaagatgtcggagtttaagtcgtcggttggttggaaggcgttttttttgtggaggtcgatagtggtagggagggggtcggggacggtttcgcggagggatttgacgtatacggagtcgtagtataggttttatttagcggcgttggttggagttgagatggaagttagtttttatgtagtagaaatatgaaataaatgaagtaaaattgtttagagaggggaaatgttttaaggatgggttttatttacgcgcgtatatagatatatacgtagagagtatttttacgttgggtaagttcgggatcgcgttatttttttcgagttgaatgatag

SEQ ID NO: 12 - DNA, 2050 bp, Artificial Sequence
  Synthetic Sequence
ttattaaatttttggttttagggaattataggttttatatttttgtaaagatgtgttttagtaaatgtaagatgaagtattgattaattattattatatattagaatttttttaataaaaaatttaggattgaatttaaggaaaataaaataaatgatagagggggaaaaaataatgttttgttatggtattgtagtggttggatagtttgtttgtgaaatgttaatgttattttggagtagttagttattagttattgtgtagggtaggagaaagttgagttggttgtgtgtgtagagtgagtaagtgaatgagtgagtgtgttttttttttttgtgttgtttttgttgttgttgatttttgtgtgtttttgtgtttgtatggatgtgtgtgttggtttttttttttttggttttgtattgtataatatttatgatgtatttttatttttagtatattaataaaatattataaataaattgtttgtagtttttgtggttttgaagtatgagtatttttggttattggtttttgtagattttgtgttggttttttaattttttttatggtttttggagtttttatgttttagggttaagtttttgtttttgtaaaaaattgtgggaaatttaatttttatttgatttagggaaaagtttttttgttttgtgatgtgaatgttttattagattttggtaggttttgggatgtttttttttggtttagttggtgtgttggggtagtagttgtggggagaggatattttgtgagtttggtttttggtttttatttgtttgttttttttttttgtttgttttgtgttttggtggggattttgagttttgtgttttgtgttttgttggtggtggtagtggtggtgtagtggtttagaggtggttgtgtggtggtgggatgtgttgggttgttgttgtttttattttttttttggttattttgtggattagtatttgtaataataaagtggggagagttgagttttgtgttttgggttgtggttttgttgttgttgttttttttttttatgtttgtgagtggtgagtgttggtttggtttgtgttgtgtgttgttgtatttgagttgtttgttgatagggtttattttttaggaattgggtgatgaggaagtagagttttgtgtgggagagagggatggggagatatataggttgagtggttgggtggtggggggtgggggattgtgggtggaattgtttggtgttagtggttttggtagttttttgatttatttgattgtagttttgagaggtttttttttttataagatatgtttatgggttattttagggttgttgatgggatattttgttgttgaggggaagtggggatggtgttgttgtttgttttatagttgttagtgtgtgtttatgagttgagttttggttttattatttaagtaatggtggtggaggtggaggaggaggaggaggaaataataatttttaggtagtgattatggttgtggttgtttttgttgtggatttttttgttgtagaaaggagtttgttgtttttgtggtttagggtttggttttggttttggtttgtgtttttgttttttggtgttaaaaaaggagtgtttttgattttttgtttttagtgttttgtatgtggtatagtgagatttttatttgtttttttttgtagggtgtgtgatttatttatttatttttagttggagaagatgttggagtttaagttgttggttggttggaaggtgttttttttgtggaggttgatagtggtagggagggggttggggatggttttgtggagggatttgatgtatatggagttgtagtataggttttatttagtggtgttggttggagttgagatggaagttagtttttatgtagtagaaatatgaaataaatgaagtaaaattgtttagagaggggaaatgttttaaggatgggttttatttatgtgtgtatatagatatatatgtagagagtatttttatgttgggtaagtttgggattgtgttattttttttgagttgaatgatag

SEQ ID NO: 13 - DNA, 1800 bp, Homo sapiens
gggttcacggacttctctgagcggcttgaccgagaacccccagggctacagcgagacgctccacttgtctccttttgggttctccggcagaaggcagacatttacagtccgcttccagccccaggccggatccggggagcgcggggccagagccccaggccggatccggggagcgcggggccagcctggcggtggttttgcccgaggagcgggctcccacgcgcgagtcaggcttcctccaccaggcgcccagacctcgatgcccatgaacgctcagagtgggtccttagaaccgcatttcaacacatcttacagggctccgccaacacacacacagaacatgctccaagcagtgcccgagcgcacagggcaagcccgggagcaacccagagctgggggaggagaaaaggggactccgggagggacggggaagtagttagacaatagtcaggggaggttattcccgggacaaaaagcacctggcaaagcgcgccgaggtcagtcccttcgaatctccacatttctgacaatggcttcccagggaatttgtttaaacacccaagtggcagacagcaacaaacccgcaccacaagcaacagagtgccgtcacgggagtcgtaactactttttctttctctcttccccactagcccggcgcaggccacgcgagacgccgaagccagcaggtccccacactctccggctagaccgtgccagcctgagccccgagcaagcttcggagaggaacaatgggtgccccgcgtcccacctggcacgaactctggggacagccctccctctaccctgtgttccccacccccggacagagagagctccccacgatccttgtccccagccctagcggggacctggctcacccctcgtgagcccaggactccaggcgttcgtcggggacgccccccggccactcacctgcggagaactggccttgggccgatcccaggagaggccaaggagagaggagcaagagcgccacaaaagcaggcagcgccgccgccgcagtcgtatccatgccgagtttgggagaagtttcaagcagctttgcaaagagctgccggggggatcgccgcgaaatccacgacggaggagcgggccgggcctcgcggggtgaggacggtgagaggacagccgcccgcccgccctttttccttcttcgcggtcgccaaactacctcaggggcgaaagcgtcgccagcgtcgccggccggccgcggcggcagctctccatgctcggcggaggctgctcctgttagtcaagagttactttgctcggggagggacgggggcggagccgggggtgacgtcgccccgcgggctccgggcgcttctgaggaatgcgggctcgggcgcggcgccgccaccgcctcctgccggagagacgcgcgccagccccggccgcgcgccccgcgccgcctttgttcgcagcctgggccccgccgccagccgctgctcggagggagcgagcgagaaaggggagccggcgcagctcgctgccctgttccagaactcagaatttgagaggcgagagttcggtaagccgtgcgaccagacgaccgaggacgcgcgcagagaaacccccgccgtccgaggagaggcgtcctggggttccgtcccaaggtggaggtccccccgagcggttgctgattgaaaggggagtgtggaagcgccctgaggaaacacccgcctgccttgaaaactccccgttccttattcttgtaataaggaataagacaccccacccccatcgaaaacttgtcccttattcatagaataaataaaa

SEQ ID NO: 14 - DNA, 1800 bp, Artificial Sequence
  Synthetic Sequence
gggtttacggatttttttgagcggtttgatcgagaatttttagggttatagcgagacgttttatttgtttttttttgggtttttcggtagaaggtagatatttatagttcgtttttagttttaggtcggattcggggagcgcggggttagagttttaggtcggattcggggagcgcggggttagtttggcggtggttttgttcgaggagcgggtttttacgcgcgagttaggttttttttattaggcgtttagatttcgatgtttatgaacgtttagagtgggtttttagaatcgtattttaatatattttatagggtttcgttaatatatatatagaatatgttttaagtagtgttcgagcgtatagggtaagttcgggagtaatttagagttgggggaggagaaaaggggatttcgggagggacggggaagtagttagataatagttaggggaggttattttcgggataaaaagtatttggtaaagcgcgtcgaggttagttttttcgaatttttatatttttgataatggttttttagggaatttgtttaaatatttaagtggtagatagtaataaattcgtattataagtaatagagtgtcgttacgggagtcgtaattatttttttttttttttttttttattagttcggcgtaggttacgcgagacgtcgaagttagtaggtttttatatttttcggttagatcgtgttagtttgagtttcgagtaagtttcggagaggaataatgggtgtttcgcgttttatttggtacgaattttggggatagttttttttttattttgtgttttttattttcggatagagagagttttttacgatttttgtttttagttttagcggggatttggtttatttttcgtgagtttaggattttaggcgttcgtcggggacgtttttcggttatttatttgcggagaattggttttgggtcgattttaggagaggttaaggagagaggagtaagagcgttataaaagtaggtagcgtcgtcgtcgtagtcgtatttatgtcgagtttgggagaagttttaagtagttttgtaaagagttgtcggggggatcgtcgcgaaatttacgacggaggagcgggtcgggtttcgcggggtgaggacggtgagaggatagtcgttcgttcgtttttttttttttttcgcggtcgttaaattattttaggggcgaaagcgtcgttagcgtcgtcggtcggtcgcggcggtagttttttatgttcggcggaggttgtttttgttagttaagagttattttgttcggggagggacgggggcggagtcgggggtgacgtcgtttcgcgggtttcgggcgtttttgaggaatgcgggttcgggcgcggcgtcgttatcgttttttgtcggagagacgcgcgttagtttcggtcgcgcgtttcgcgtcgtttttgttcgtagtttgggtttcgtcgttagtcgttgttcggagggagcgagcgagaaaggggagtcggcgtagttcgttgttttgttttagaatttagaatttgagaggcgagagttcggtaagtcgtgcgattagacgatcgaggacgcgcgtagagaaattttcgtcgttcgaggagaggcgttttggggtttcgttttaaggtggaggttttttcgagcggttgttgattgaaaggggagtgtggaagcgttttgaggaaatattcgtttgttttgaaaatttttcgttttttatttttgtaataaggaataagatattttatttttatcgaaaatttgttttttatttatagaataaataaaa

SEQ ID NO: 15 - DNA, 1800 bp, Artificial Sequence
  Synthetic Sequence
gggtttatggatttttttgagtggtttgattgagaatttttagggttatagtgagatgttttatttgtttttttttgggttttttggtagaaggtagatatttatagtttgtttttagttttaggttggatttggggagtgtggggttagagttttaggttggatttggggagtgtggggttagtttggtggtggttttgtttgaggagtgggtttttatgtgtgagttaggttttttttattaggtgtttagattttgatgtttatgaatgtttagagtgggtttttagaattgtattttaatatattttatagggttttgttaatatatatatagaatatgttttaagtagtgtttgagtgtatagggtaagtttgggagtaatttagagttgggggaggagaaaaggggattttgggagggatggggaagtagttagataatagttaggggaggttatttttgggataaaaagtatttggtaaagtgtgttgaggttagtttttttgaatttttatatttttgataatggttttttagggaatttgtttaaatatttaagtggtagatagtaataaatttgtattataagtaatagagtgttgttatgggagttgtaattatttttttttttttttttttttattagtttggtgtaggttatgtgagatgttgaagttagtaggtttttatattttttggttagattgtgttagtttgagttttgagtaagttttggagaggaataatgggtgttttgtgttttatttggtatgaattttggggatagttttttttttattttgtgttttttatttttggatagagagagttttttatgatttttgtttttagttttagtggggatttggtttattttttgtgagtttaggattttaggtgtttgttggggatgttttttggttatttatttgtggagaattggttttgggttgattttaggagaggttaaggagagaggagtaagagtgttataaaagtaggtagtgttgttgttgtagttgtatttatgttgagtttgggagaagttttaagtagttttgtaaagagttgttggggggattgttgtgaaatttatgatggaggagtgggttgggttttgtggggtgaggatggtgagaggatagttgtttgtttgttttttttttttttttgtggttgttaaattattttaggggtgaaagtgttgttagtgttgttggttggttgtggtggtagttttttatgtttggtggaggttgtttttgttagttaagagttattttgtttggggagggatgggggtggagttgggggtgatgttgttttgtgggttttgggtgtttttgaggaatgtgggtttgggtgtggtgttgttattgttttttgttggagagatgtgtgttagttttggttgtgtgttttgtgttgtttttgtttgtagtttgggttttgttgttagttgttgtttggagggagtgagtgagaaaggggagttggtgtagtttgttgttttgttttagaatttagaatttgagaggtgagagtttggtaagttgtgtgattagatgattgaggatgtgtgtagagaaatttttgttgtttgaggagaggtgttttggggttttgttttaaggtggaggtttttttgagtggttgttgattgaaaggggagtgtggaagtgttttgaggaaatatttgtttgttttgaaaattttttgttttttatttttgtaataaggaataagatattttatttttattgaaaatttgttttttatttatagaataaataaaa

SEQ ID NO: 16 - DNA, 20 bp, Artificial Sequence
  Primer
acgcgtgcgtagtttgggtc

SEQ ID NO: 17 - DNA, 22 bp, Artificial Sequence
  Primer
ctaccgatttctcgacaacgcc

SEQ ID NO: 18 - DNA, 22 bp, Artificial Sequence
  Primer
gaggagtagaacgaggagcggt

SEQ ID NO: 19 - DNA, 21 bp, Artificial Sequence
  Primer
tacaaacgacccgaaacgaaa

SEQ ID NO: 20 - DNA, 23 bp, Artificial Sequence
  Primer
tggtttatcgtgtataagggcgt

SEQ ID NO: 21 - DNA, 22 bp, Artificial Sequence
  Primer
cgactcctctctcaatccgaaa

SEQ ID NO: 22 - DNA, 22 bp, Artificial Sequence
  Primer
tagcgtttcgtacgcggtatag

SEQ ID NO: 23 - DNA, 22 bp, Artificial Sequence
  Primer
caaccgacgacttaaactccga

SEQ ID NO: 24 - DNA, 22 bp, Artificial Sequence
  Primer
tcgtagtttgggtttcgtcgtt

SEQ ID NO: 25 - DNA, 22 bp, Artificial Sequence
  Primer
gacttaccgaactctcgcctct

SEQ ID NO: 26 - DNA, 22 bp, Artificial Sequence
  Primer
gttaggtgagagagagttgtgg

SEQ ID NO: 27 - DNA, 25 bp, Artificial Sequence
  Primer
ctactcctactataaaaactactcc

SEQ ID NO: 28 - DNA, 21 bp, Artificial Sequence
  Primer
aactcgaaccgaacctatccg

SEQ ID NO: 29 - DNA, 25 bp, Artificial Sequence
  Primer
gattagtattagggtattatttggt

SEQ ID NO: 30 - DNA, 23 bp, Artificial Sequence
  Primer
actcctctaaatactcccatctc

SEQ ID NO: 31 - DNA, 21 bp, Artificial Sequence
  Primer
atcgtgtataagggcgtcgag

SEQ ID NO: 32 - DNA, 25 bp, Artificial Sequence
  Primer
ggaggttgtttttgttagttaagag

SEQ ID NO: 33 - DNA, 26 bp, Artificial Sequence
  Primer
cctctcaaattctaaattctaaaaca

SEQ ID NO: 34 - DNA, 19 bp, Artificial Sequence
  Primer
tctaaaacaaaacaacgaa

SEQ ID NO: 35 - DNA, 21 bp, Artificial Sequence
  Primer
gggtcgttcgtgggttatttc

SEQ ID NO: 36 - DNA, 20 bp, Artificial Sequence
  Primer
cgcctccaaatacgcactac

SEQ ID NO: 37 - DNA, 23 bp, Artificial Sequence
  Primer
tacgacgatgtcggcggacgtta

SEQ ID NO: 38 - DNA, 21 bp, Artificial Sequence
  Primer
gctttgcgctacgatgctatg

SEQ ID NO: 39 - DNA, 20 bp, Artificial Sequence
  Primer
actctgccatccgctgcttt

SEQ ID NO: 40 - DNA, 22 bp, Artificial Sequence
  Primer
taagattcgagctcgcgctcgg

SEQ ID NO: 41 - DNA, 22 bp, Artificial Sequence
  Primer
tcgtagtttgggtttcgtcgtt

SEQ ID NO: 42 - DNA, 23 bp, Artificial Sequence
  Primer
gacttaccgaactctcgcctctc

SEQ ID NO: 43 - DNA, 23 bp, Artificial Sequence
  Primer
ttcggagggagcgagcgagaaag